SEQUENCE LISTING (112 sequences)

SEQ ID NO: 1 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 2 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 3 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 4 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 5 - DNA, 1000 bp, Arabidopsis thaliana
  misc_feature (1)..(1000): Ceres Promoter PT0613
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 6 - DNA, 351 bp, Arabidopsis thaliana
  misc_feature (1)..(351): Ceres Promoter PT0625
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 7 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 8 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 9 - DNA, 998 bp, Arabidopsis thaliana
  misc_feature (1)..(998): Ceres Promoter PT0660
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 10 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 11 - DNA, 999 bp, Arabidopsis thaliana
  misc_feature (1)..(999): Ceres Promoter PT0672
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 12 - DNA, 1000 bp, Arabidopsis thaliana
  misc_feature (1)..(1000): Ceres Promoter PT0676
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 13 - DNA, 998 bp, Arabidopsis thaliana
  misc_feature (1)..(998): Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 14 - DNA, 1000 bp, Arabidopsis thaliana
  misc_feature (1)..(1000): Ceres Promoter PT0683
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 15 - DNA, 1000 bp, Arabidopsis thaliana
  misc_feature (1)..(1000): Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 16 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 17 - DNA, 1000 bp, Arabidopsis thaliana
  misc_feature (1)..(1000): Ceres Promoter PT0708
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 18 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 19 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 20 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 21 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 22 - DNA, 1000 bp, Arabidopsis thaliana
  misc_feature (1)..(1000): Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 23 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 24 - DNA, 763 bp, Arabidopsis thaliana
  misc_feature (1)..(763): Ceres Promoter PT0837
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 25 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 26 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 27 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 28 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 29 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 30 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 31 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 32 - DNA, 999 bp, Arabidopsis thaliana
  misc_feature (1)..(999): Ceres Promoter YP0019
gatataagtagaatcattttttgccgccgtttctcgctaacacaccgaaaactgaatcaaatctcctagctcttctacgcaaaatcgagtgcatcgacaatggcggaacgtggtgtcgaacgtggtggagatcgcggcgatttcggacgtggattcggtggtcgcggcggtggaagaggtggtccgagaggtcgtggtcgccgtgcaggtcgtgctccagaggaggagaaatgggtgccagtgactaagcttggtcgtctcgtaaaggaaggtaagatcacaaagattgagcagatctacctccattctctcccagtcaaggagtaccagatcatagatttactcgtcggtccttcattgaaagacgaagtgatgaaaatcatgccggttcaaaaacaaaccagagccggtcagagaacgagattcaaggccttcatcgtcgtcggagatagtaacggtcacgtcggattaggagtcaaatgctccaaggaagttgcgacggcgatcagaggcgcgatcattctcgcgaaattgtctgtggttccgatacgaagaggttattggggtaacaagattggaaaaccacatacggttccgtgtaaggtaaccgggaaatgtggatctgttactgtacgtatggttccagctccgagaggttctggtattgtggcggctagagttcctaagaaggttcttcaattcgctggaattgatgatgtctttacttcttctagaggatccaccaaaactcttggaaacttcgtcaaggtatgtactttcacaatggctgttttggtttgatgaactctgaattaggcagtgaaaaagtaatcattaccagttaagtgaatttatattgaagattaggatttagctgattgtattggtttgagcatgtgagtttgtgtttaagattgcttgaattgaaatgctttaggttgtttgattacgctaaattctgactaatgtaattcaaattgttgttgtttttttttggtc

SEQ ID NO: 33 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 34 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 35 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 36 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 37 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 38 - DNA, 1024 bp, Arabidopsis thaliana
  misc_feature (1)..(1024): Ceres Promoter YP0092
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaat

SEQ ID NO: 39 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 40 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 41 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 42 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 43 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 44 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 45 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 46 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 47 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 48 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 49 - DNA, 1000 bp, Arabidopsis thaliana
  misc_feature (1)..(1000): Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 50 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 51 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 52 - DNA, 1004 bp, Arabidopsis thaliana
  misc_feature (1)..(1004): Ceres Promoter YP0128
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 53 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 54 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 55 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 56 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 57 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 58 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 59 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 60 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 61 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 62 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 63 - DNA, 999 bp, Arabidopsis thaliana
  misc_feature (1)..(999): Ceres Promoter YP0275
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 64 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 65 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 66 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 67 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 68 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 69 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 70 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 71 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 72 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 73 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 74 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 75 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 76 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 77 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 78 - DNA, 1024 bp, Arabidopsis thaliana
  misc_feature (1)..(1024): Ceres Promoter PR0924
atctataacgagttaacatgttgccagtttgaatcaagaagcttggatgatgaatgaatggatcggtttgtggtacaattcttaaaattgtagtagaggagacagagaaaaaacatgataagactttggtatttacaacttgacggagacaagacagtaagccaaatctgtcacaaaaacactcaaactcttttctcagtgttttgagtttaaagagagacttattcacttcccctttcgtaacacttatttgtctcccaaccaaacagtttctgtcctttcccttgtcctcccacgtgcatctttatatctcatgacttttcgtttctagatcttgaataatgtcttagtggattaggtttgttgtcggtaaattaggtgaccgtttttttcttatatttggaagatcgcgggatgaagcagatactgagtttcagggcatacacacctaatttgaaaatcattgttagtccaatttcactttaatcttgtttacaaaaaaattgatctgaaaatgttgatgggataagtaaaaatgtaagttttgctagtagtcatgatataataatagcaaaaccagatcaattttgagcaaaaggaagaaacaaaaaacagatcgatcccacgagcaagactaagtgtaaagtggttcccacaagagccatatggatatggtccttcaacttttaaagcccattacttcagtggtcgacccgacattacgccacgagtagtcacgcacgcacgactccgttcacgtgacattcacgttgatatttccccctctactctcttctgcttggttgatctaaaaaacatgaagagaccaacctaatttcatattaatatatgatatagacttcatactcaacagtcactttcgtaatccaaatccatatcttacgaaattagttcttaataaaggttgtggattaagttataatattgtgttaagagttaagacacagcatataaccttgtaccaacagtgctttattcttaaatggaaacaaaacatatgtca

SEQ ID NO: 79 - DNA, 857 bp, Arabidopsis thaliana
  misc_feature (1)..(857): Ceres Promoter PD1367
ttggaattaattctgcggccatggggctgcaggaattcgatggcccgatcggccacagttttcttttctcatcttacaacaagtttccaggaggatagagacataaacgaagctcnggattgtatcgttctttttnagcttttattcacatccngaaangtcctgtangttntangattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttnnctcctnctttcacttcatcacttggcttgtgaattcattaattncctgcgaaggccntgcagggccatcaccactgcagtggaacaatgaagactaatctttttctctttctcatcttttcacttctcctatcattatcctcggccgaattcagtaaaggagaagaacttttcactg

SEQ ID NO: 80 - DNA, 692 bp, Arabidopsis thaliana
  misc_feature (1)..(692): Ceres CLONE ID no. 18200
aacaccttcttctccactctcattctctctttctgacacattaactacttatccttcttgcattcttctctctctctacacccaaacaaacacacttataatatatcaagaaagaagatgtctagcagaagatcatcacgttcaagacagtcaggaagctcaagaatctctgacgatcagatttccgatcttgtttctaagctccaacacctcatccctgaacttcgccgccgccgttctgacaaggtgtcagcatctaaggtactacaagagacttgcaactacatcaggaacttacacagagaggttgatgacctcagtgaccgtttgtcggaactcttggcttcgacggacgacaacagcgccgaaacagccatcattaggagcttgcttaattattaaatccgcattacttaatctgagagctattaatcatccgtttccggccaccaaatttatcttattatgggtatcgtctgtttacttctacatcatatattatgagatatagctagggtttcgggtcattgttaggccaactcatatatttatatttaatatatggttatgtatgtatgtatgcatgttaattgtatctgagggtccagacctggcgtatagtagcctgtgtatcatgagatcctctaatatttatgattaatgacacggtccgtttccttttttactatacc

SEQ ID NO: 81 - protein, 94 aa, Arabidopsis thaliana
  misc_feature (1)..(94): Ceres CLONE ID no. 18200
MSSRRSSRSRQSGSSRISDDQISDLVSKLQHLIPELRRRRSDKVSASKVLQETCNYIRNLHREVDDLSDRLSELLASTDDNSAETAIIRSLLNY

SEQ ID NO: 82 - DNA, 1155 bp, Zea mays
  misc_feature (1)..(1155): Ceres CLONE ID no. 336524
gagtcttgacacgagctcccacacgtacactgcataaataggcgagcagcgggagagagacgaccgacgaggacgaagaggaattaagccagagcgatcgaggtggtgtagtgtagcttccgagcgaagctcctaccgttgccgccggccggagatatgtcgtcgggcggccgacgtggcaggatcagcgacgacgagatcaacgagctgatctccaagctccaggctctcctcccggaatcctcacgccgccggaacgcgagccggtcgtcggcgtcgaagcttctgaaggagacgtgcgcctacgtcaagagcctgcaccgggaggtggacgacctctcggagcggctgtcggggctcatggagaccatggacaacgacagcccccaggccgagatcatccggagcctcctccggtgactccagagtccaggttccagccatgccgcttgccgccccggtcgtccggcgcgcgcggccgccctcttctgctgcctgccatctagctagctgccgcagccagcgcaggtgcacttgagattggagaaggagaagacgacgacgtacggccgagcttgcttgttcgctcgtttatttgttacggcgacgaccttaattgtattcctttgttcttaatttgttctcctccttctcctcctcctcctccttcgggtgcgtgtttgtttccgtgttgaattagttcaagagcaagagcccttgctcagaagaaggcgaccaaaagtgctctactcgatcttctcgtgtaccacgccagggggggttagtagagtagagataatgtatggacttctattaaccaactaaaggtaccacgtactggtaggttgtagttgcagtagactactggtagtagggtgtagttttgcagtagactactgtagcagttctacattgcggatgcgtccaaagagttgttgttgttctgtagattgcatgctgctcttgtctctgtcgtcgtccttagcttacggcgttacgccaatgggcggcggacaaggagctgaacgacgaccactctgtctggtggagacaaatatatacactatcacgtgattttcctctctttagctccatcctgatgtaacttggtaatttactccatccctccgtcattttttttgcaatagttctttt

SEQ ID NO: 83 - protein, 87 aa, Zea mays
  misc_feature (1)..(87): Ceres CLONE ID no. 336524
MSSGGRRGRISDDEINELISKLQALLPESSRRRNASRSSASKLLKETCAYVKSLHREVDDLSERLSGLMETMDNDSPQAEIIRSLLR

SEQ ID NO: 84 - DNA, 712 bp, Arabidopsis thaliana
  misc_feature (1)..(712): Ceres CLONE ID no. 4734
aatatttgaagtgtattcaaaaccccaaaacacttttctcattctcttctctattttcttcttgctctctagtttttctttcttcttggtcgtttcctttcagcataaaaaccttataaaatcataaaagcttacacctacttgccacatagacatagccgatctcattatatctctatttctatttctcaatagaacttgtttgagctagtgtgagagaagtaaagaaagagagaagaatccacaacttagttagggtcttttcttgccacattgttgaacatgtcgaacagaagatcaaggcaatcttcaagtgctccaaggatctccgataatcaaatgattgacctcgtatctaagctccgtcaaattttgccggagattggtcaacgacgtcgttctgataaggcatcagcctcgaaagtattgcaagagacatgcaattacatacgaaatttgaacagagaagttgacaatctgagcgagcgtttgtctcagcttctcgaatctgtcgatgaagatagccctgaagccgccgttattagaagcctactcatgtaatcttttttgttcttttgtttgtttttgacaagcctatccatgtaatcttaaatgatcgctctataataattatatttttaacataatcgtcttattatgtaaaattcaaagagatgggcttgatctttaatgacatacgaatttcataggg

SEQ ID NO: 85 - protein, 92 aa, Arabidopsis thaliana
  misc_feature (1)..(92): Ceres CLONE ID no. 4734
MSNRRSRQSSSAPRISDNQMIDLVSKLRQILPEIGQRRRSDKASASKVLQETCNYIRNLNREVDNLSERLSQLLESVDEDSPEAAVIRSLLM

SEQ ID NO: 86 - DNA, 557 bp, Arabidopsis thaliana
  misc_feature (1)..(557): Ceres CLONE ID no. 519
atactatcaacttttctctatctatctctctctcttctttttccggcataacttctgtgttaccctaaactccataacctgtttcatcgataaagtgcctttgcttctatctctgtcactcttactacttgttgaacaatattctacaaaaaaatgtcgggaagaagatcacgttcgaggcaatcatcaggaacttcaaggatctcagaagatcaaatcaatgatctgattatcaagttgcaacagcttcttcctgagctcagggacagtcgtcgttccgacaaggtttcagcagcgagggtgttacaagatacgtgcaactacatacggaatctgcatagagaggttgatgatctaagtgagaggctatctgagttactagcaaactcagacactgcacaagctgctttaatcagaagcttacttacccaataattcctatctatctttttcttcttcttcttttttttgtttactataataataataatagtttgcgggtttttttttctatagatgttgatgaccttataaacgtttaatgatacgagttcgtc

SEQ ID NO: 87 - protein, 93 aa, Arabidopsis thaliana
  misc_feature (1)..(93): Ceres CLONE ID no. 519
MSGRRSRSRQSSGTSRISEDQINDLIIKLQQLLPELRDSRRSDKVSAARVLQDTCNYIRNLHREVDDLSERLSELLANSDTAQAALIRSLLTQ

SEQ ID NO: 88 - DNA, 771 bp, Glycine max
  misc_feature (1)..(771): Ceres CLONE ID no. 560681
atatatcttagccttttctctccctcccttctcccatattatatagcttgtcttttatttcttagactccatccattcttctccccaattgaatcttctttattttgtttcttcactgtctcgttatggctatagttttgcatagtaaataaactgaactgaagctatctatatagcagcaagtgttgatttaattacttactttagacaataattatattaattacaccaatttataagctctttatctatctatctagctagggaaaattaaaatgtctagcagaaggtccaggcagcaatctgcatccacaaggatctccgatgaccaaatcatcgacctcgtttcaaagttgcgtcaacttgttcctgagattcgcgataggcgctctgacaaggtatcagcatctaaggtcctacaagagacctgcaactacatcagaagcttacacagagaagtggatgacttaagcgaacgactgtctcagttgttggccacaatcgatgctgatagccctgaagctgccatcattaggagcctaattaactaataatatatattaagcgcaagtaatcatctaattttcctatattcaaggagatatattataagagtgtattaatttcttcttytaaattaggtggcatagagtgcagtttgaggtgcgtacgtacgtccttccaatatattatagtacatggcaggaatggtgcacttgtgtaagttaaaggtttttgcaataagaactaaggactctctgtattatggcg

SEQ ID NO: 89 - protein, 91 aa, Glycine max
  misc_feature (1)..(91): Ceres CLONE ID no. 560681
MSSRRSRQQSASTRISDDQIIDLVSKLRQLVPEIRDRRSDKVSASKVLQETCNYIRSLHREVDDLSERLSQLLATIDADSPEAAIIRSLIN

SEQ ID NO: 90 - DNA, 697 bp, Glycine max
  misc_feature (1)..(697): Ceres CLONE ID no. 560948
atgtgttttatttcctacttcccaacccataaccattaattcttaattagtttcctcttgctttcttctccttctatattattactagtacaacttatcatacacatatacctctggttatagcagcaaactaggctatagcttgcactttgaagatatttatacacagaccaagtacaacaccaagctctagctagctagggacatgtctagccgaagatccagacaacattcagggtctacaaggatctccgatgaccaaatcatcgaacttgtttccaaattgcgccaacttgttcctgagattcgcaataggcgatctgataaggtttcagcgtcaaaggtcctacaagagacctgcaactacatcagaggcttgcacagagaggtgagtgacttgagcgagcgactgtctcagttgttgaccacaattgatgctgatagtgctgaggctggaatcattaggagcctacttaatcaatgagagagtgttatgattttttatttattcaaagagagtgttaattaaattataattatgattattataagagtattgtacttcattctaggtgtgctgagagcctgggagagttcagtttttgaggctgtacctcccatatggcaggtagggcattttcataactagttgtttttctcttttttccaataaaaattcaagtgcttgtaccagctg

SEQ ID NO: 91 - protein, 92 aa, Glycine max
  misc_feature (1)..(92): Ceres CLONE ID no. 560948
MSSRRSRQHSGSTRISDDQIIELVSKLRQLVPEIRNRRSDKVSASKVLQETCNYIRGLHREVSDLSERLSQLLTTIDADSAEAGIIRSLLNQ

SEQ ID NO: 92 - DNA, 728 bp, Glycine max
  misc_feature (1)..(728): Ceres CLONE ID no. 653656
acatgcaacttgtcttaatttctttctcgatccccaacatcactagctagctccttttgtacacactctacaaccccacctagctacatcacttaattagttttcccatatctataaccaatttcaaattctcacccttaactagctagctatatttcataactgattattaccaactcactacatattattggctaggattcaccattagacttaaaagtagttgatttattatatatataagatgtctagcaggaggtcacggtcaaggcaaacaagtagttcaaggaatatcaccgatgatcagatcaatgatcttgtctccaagttgcaacagcttcttccagagattcgcgataggcgctctgacaaggtttcagcttccaaggtgttgcaagagacatgcaactatattagaagcttacacagggaagtggatgacctaagcgagcgtttatctgagctcttggctacaactgacacagcacaagctgcaataattagaaatctactaatgcaatagatcggtgcagtagttaatttatcgcataattcatagttagcacttcagtacttgtgaaccgatccagtcagtagtcgcgtatttcttattctctttttgtttcactttttttttctggtttttgtccactaatatgcatgattactgcttttgcaaagcccattttcctaagatattaaataaaagtctgagtttgcgctttgct

SEQ ID NO: 93 - protein, 92 aa, Glycine max
  misc_feature (1)..(92): Ceres CLONE ID no. 653656
MSSRRSRSRQTSSSRNITDDQINDLVSKLQQLLPEIRDRRSDKVSASKVLQETCNYIRSLHREVDDLSERLSELLATTDTAQAAIIRNLLMQ

SEQ ID NO: 94 - DNA, 701 bp, Triticum aestivum
  misc_feature (1)..(701): Ceres CLONE ID no. 733804
atgcacaacttgtcttccctctcttccaacaccacttcttcttagttcctctccgtccctgtctgccaccacttctgtctctcaaacttgtctcactccaaccataaaccctcactgtcttgggctctctctgccaagcatccaattcctaagtacatccgatcactcacatttgcagtgatgtcgagccgtaggtcaaggtcaaggcagtccggctcgtcgaggatcaccgacgagcaaatcagcgaccttgtctccaagttgcaggacctccttcccgaggcgcgtctccggggcaatgatagagtgccatcttcaagggtgctgcaggagacgtgcacctacatcaggagcttgcaccgggaggtggacgacctgagcgagaggctgtcggagctgctggcgacctcggacatgagcagcgcgcaagcggccatcatccgcagcttgctgatgtagagccggctcccatgcagtgcgcaggcgcctcgtcgctgtcttgctgagcgcacaagcctgaatttgagcgtttgtagcctagggagcgatctttaattagtaccggagttgcaggttacctacttaatccgcgtgtgtgcgctgtcgtcgtgtcatcatcgtyttaatcaggcccatcttttttgtgtgtgtacttaaatcaagtcgttaaatcaaaccccgcccgtggttggtgcaagttg

SEQ ID NO: 95 - protein, 92 aa, Triticum aestivum
  misc_feature (1)..(92): Ceres CLONE ID no. 733804
MSSRRSRSRQSGSSRITDEQISDLVSKLQDLLPEARLRGNDRVPSSRVLQETCTYIRSLHREVDDLSERLSELLATSDMSSAQAAIIRSLLM

SEQ ID NO: 96 - DNA, 545 bp, Arabidopsis thaliana
  misc_feature (1)..(545): Ceres CLONE ID no. 8607
ctccctttctttcgacaagcacaaacaaagccatcaagagaagaaagccttttcttggattcacatatatataagaatattttttcaaatcaaacatgtcttctagcagaaggtcgagacaagcaagctcatcatcaagaattagcgatgaccagatcactgatctcatctcaaagctccgacagtccattccggagattcgccagaaccgtcgttccaacacggtatcagcgtcgaaagtgttacaagagacttgcaactacataagaaacttgaacaaggaagccgatgacctcagtgatcgattgactcagcttctggaatccattgatcctaatagcccacaagccgcagttattaggagcttgattaatggataattaagatataaattgattagttgtgctttatatatataagcttaaaatctcgttgggaggttgatccatcagggtgttgcataattatatatctattttatgtttcttatatattatttacaatcctatctagttagggttcatattttgacccttttttggttt

SEQ ID NO: 97 - protein, 94 aa, Arabidopsis thaliana
  misc_feature (1)..(94): Ceres CLONE ID no. 8607
MSSSRRSRQASSSSRISDDQITDLISKLRQSIPEIRQNRRSNTVSASKVLQETCNYIRNLNKEADDLSDRLTQLLESIDPNSPQAAVIRSLING

SEQ ID NO: 98 - protein, 91 aa, Oryza sativa subsp. japonica
  misc_feature (1)..(91): Public GI no. 78708592
MSRRSRSRASSAARITDEQIGDLVSKLQALLPEARLRSNDRVPSARVLQETCSYIRSLHREVDDLSERLAELLAAADVSTAQAAVIRGLLM

SEQ ID NO: 99 - protein, 92 aa, Glycine max
  misc_feature (1)..(92): Ceres CLONE ID no. 663844
MSSRRSRSRQTSSSRNITDDQINDLVSKLQQLLPEIRDRRSDKVSASKVLQETCNYIRSLHREVGDLSERLSELLDTTDTAQAAIIRNLLMQ

SEQ ID NO: 100 - DNA, 276 bp, Populus balsamifera subsp. trichocarpa
  misc_feature (1)..(276): Ceres GDNA ANNOT ID no. 1468218
atgtctagccgaaggtcacgatcaaggcaatcaagtagttcaagaatcagtgatgatcagatccttgatcttgttacaaagttgcaacaacttcttcctgagattcgtaacaggcgttctgacaaggtttcggctgccaagatcttgcaggagacatgcaactatattaaaagcttgcatagagaggttggtgatcttagcgagcggctgtctgagctattggaaacaactgatacagcccaagctgcaataatcaggaacttacttatgcaatag

SEQ ID NO: 101 - protein, 91 aa, Populus balsamifera subsp. trichocarpa
  misc_feature (1)..(91): Ceres GDNA ANNOT ID no. 1468218
MSSRRSRSRQSSSSRISDDQILDLVTKLQQLLPEIRNRRSDKVSAAKILQETCNYIKSLHREVGDLSERLSELLETTDTAQAAIIRNLLMQ

SEQ ID NO: 102 - protein, 91 aa, Triticum aestivum; identical to SEQ ID NO: 89
  misc_feature (1)..(91): Ceres CLONE ID no. 703180

SEQ ID NO: 103 - protein, 86 aa, Brassica napus
  misc_feature (1)..(86): Ceres CLONE ID no. 945972
MSSRRSSCSRQSGSSRISDDQISDLVTKLQHLIPELRRRRSDXVSASKVLQETCNYIRNLHREVDDLSDRLSEFLASTDDNSAEXA

SEQ ID NO: 104 - DNA, 273 bp, Populus balsamifera subsp. trichocarpa
  misc_feature (1)..(273): Ceres GDNA ANNOT ID No. 1530225
atgtctagcagaaggccaaggcaatctagcgttccaaggatcactgatgatcagatcatcgaccttgtctccaaattacgccagcttctccctgagattagtcaaaggcgctccgataaggtatcagcttccaaggtcctacaagagacttgcaattatatcaggaacttgcacagggaggttgatgacttaagtgagcgattgtctcagcttttggcaacaattgatgctgatagtcctgaagcagcgataataaggagtttaattatgtaa

SEQ ID NO: 105 - protein, 90 aa, Populus balsamifera subsp. trichocarpa
  misc_feature (1)..(90): Ceres GDNA ANNOT ID No. 1530225
MSSRRPRQSSVPRITDDQIIDLVSKLRQLLPEISQRRSDKVSASKVLQETCNYIRNLHREVDDLSERLSQLLATIDADSPEAAIIRSLIM

SEQ ID NO: 106 - protein, 92 aa, Arabidopsis thaliana
  misc_feature (1)..(92): Public GI no. 22331645
MSSRKSRSRQTGASMITDEQINDLVLQLHRLLPELANNRRSGKVSASRVLQETCSYIRNLSKEVDDLSERLSQLLESTDSAQAALIRSLLMQ

SEQ ID NO: 107 - DNA, 273 bp, Populus balsamifera subsp. trichocarpa
  misc_feature (1)..(273): Ceres GDNA ANNOT ID no. 1449794
atgtctagcagaaggtcaaggcagtctagtgttccaaggatcactgatgatcaaatcatccaccttgtctccaaattacgccagcttctccctgagattcgtcaaaggcgctccgataaggtatcagcttctaaggtcctacaagaaacttgcaactatatcaagaacttgcatagggaggttgatgatttaagtgagcgattgtctcagcttttggcaacaattgattctgatagtcctgaagctgagataataaggagtttaattatgtaa

SEQ ID NO: 108 - protein, 90 aa, Populus balsamifera subsp. trichocarpa
  misc_feature (1)..(90): Ceres GDNA ANNOT ID no. 1449794
MSSRRSRQSSVPRITDDQIIHLVSKLRQLLPEIRQRRSDKVSASKVLQETCNYIKNLHREVDDLSERLSQLLATIDSDSPEAEIIRSLIM

SEQ ID NO: 109 - protein, 88 aa, Oryza sativa subsp. japonica
  misc_feature (1)..(88): Public GI no. 31431968
MSGRRASGRITDDEINELISKLQSLLPESSRRRGATSRSPATKLLKEMCSYIKSLHREVDDLSERLSELMATMDSNSPQADIIRSLLR

SEQ ID NO: 110 - protein, 88 aa, Oryza sativa subsp. japonica
  misc_feature (1)..(88): Public GI no. 50912765
MSSRRSSRGSISEEEINELISKLQSLLPNSRRRGSSQASTTKLLKETCNYIKSLHREVDDLSDRLSDLMATMDHNSPGAEIIRSILRS

SEQ ID NO: 111 - protein, 90 aa, Zea mays
  misc_feature (1)..(90): Ceres CLONE ID no. 486120
MSSRRSSSHGNISEDEMNELVSKLQALLPSSRRRRGSGQASTAKLLKETCSYIKSLQREVDDLSDRLSDLLSTMDHNSPAAEIIRSILRS

SEQ ID NO: 112 - protein, 89 aa, Zea mays
  misc_feature (1)..(89): Ceres CLONE ID no. 503296
MSSRRPSSRGNISEDEINELISKLQALLPSSRRRGSGQASTTKLLKETCSYIKSLHREVDDLSDRLSDLMATMDHNSPGAEIIRSILRS

(19) United States
(12) Patent Application Publication
(10) Pub. No.: US 2010/0310753 A1
(43) Pub. Date: Dec. 9, 2010

(54) NUCLEOTIDE SEQUENCES AND CORRESPONDING POLYPEPTIDES CONFERRING MODULATED PLANT GROWTH RATE AND BIOMASS IN PLANTS

(76) Inventors: NICKOLAI ALEXANDROV, THOUSAND OAKS, CA (US); VYACHESLAV BROVER, SIMI VALLEY, CA (US); PETER MASCIA, THOUSAND OAKS, CA (US); KENNETH FELDMANN, NEWBURY PARK, CA (US); RICHARD SCHNEEBERGER, VAN NUYA, CA (US); GREG NADZAN, WOODLAND HILLS, CA (US)

Correspondence Address: BIRCH STEWART KOLASCH & BIRCH, PO BOX 747, FALLS CHURCH, VA, 22040-0747, US

(21) Appl. No.: 12/776,319

(22) Filed: May 7, 2010

Related U.S. Application Data
(62) Division of application No. 11/324,093, filed on Dec. 29, 2005, now Pat. No. 7,803,983.
Application No. 11/324,093 is a continuation-in-part of application No. 11/172,740, filed on Jun. 30, 2005, now Pat. No. 7,396,979.
(60) Provisional application No. 60/583,621, filed on Jun. 30, 2004.
(60) Provisional application No. 60/584,826, filed on Jul. 1, 2004.
(60) Provisional application No. 60/584,800, filed on Jun. 30, 2004.

(51) Int. Cl. A01H 1/00; C07H 21/04; C12N 15/63; C12N 5/10; A01H 5/00; C12Q 1/68; A23L 1/212; A23K 1/14

(52) U.S. Cl. 426/635; 536/23.1; 435/320.1; 435/419; 800/295; 800/298; 435/6; 800/278; 426/615

(57) ABSTRACT

The present invention relates to isolated nucleic acid molecules and their corresponding encoded polypeptides able confer the trait of modulated plant size, vegetative growth, organ number, plant architecture, growth rate, seedling vigor and/or biomass in plants. The present invention further relates to the use of these nucleic acid molecules and polypeptides in making transgenic plants, plant cells, plant materials or seeds of a plant having plant size, vegetative growth, organ number, plant architecture, growth rate, seedling vigor and/or biomass that are altered with respect to wild type plants grown under similar conditions

### CROSS-REFERENCE TO RELATED APPLICATION

This application is a Divisional of co-pending application Ser. No. 11/324,093 filed on Dec. 29, 2005 and for which priority is claimed under 35 U.S.C. §120. Application Ser. No. 11/324,093 is a Continuation-In-Part of application Ser. No. 11/172,740, U.S. Pat. No. 7,396,979 filed on Jun. 30, 2005, the entire contents of which are hereby incorporated by reference and for which priority is claimed under 35 U.S.C. §120. Application Ser. No. 11/172,740 claims priority on Application Nos. 60/583,621 filed on Jun. 30, 2004, 60/584,826 filed on Jun. 30, 2004, and 60/584,800 filed on Jun. 30, 2004 under 35 U.S.C §119; the entire contents of all of which are hereby incorporated by reference.

### FIELD OF THE INVENTION

The present invention relates to isolated nucleic acid molecules and their corresponding encoded polypeptides able to modulate plant growth rate, vegetative growth, organ size, architecture seedling vigor and/or biomass in plants. The present invention further relates to using the nucleic acid molecules and polypeptides to make transgenic plants, plant cells, plant materials or seeds of a plant having modulated growth rate, vegetative growth, organ number, architecture, seedling vigor and/or biomass as compared to wild-type plants grown under similar conditions.

### BACKGROUND OF THE INVENTION

Plants specifically improved for agriculture, horticulture, biomass conversion, and other industries (e.g. paper industry, plants as production factories for proteins or other compounds) can be obtained using molecular technologies. As an example, great agronomic value can result from modulating the size of a plant as a whole or of any of its organs or the number of any of its organs.

Similarly, modulation of the size and stature of an entire plant, or a particular portion of a plant, or growth rate, or seedling vigor allows production of plants better suited for a particular industry. For example, reductions in the height of specific crops and tree species can be beneficial by allowing easier harvesting. Alternatively, increasing height, thickness or organ size, organ number may be beneficial by providing more biomass useful for processing into food, feed, fuels and/or chemicals (see the US Department of Energy website for Energy Efficiency and Renewable Energy). Other examples of commercially desirable traits include increasing the length of the floral stems of cut flowers, increasing or altering leaf size and shape or enhancing the size of seeds and/or fruits. Changes in organ size, organ number and biomass also result in changes in the mass of constituent molecules such as secondary products and convert the plants into factories for these compounds.

Availability and maintenance of a reproducible stream of food and animal feed to feed animals and people has been a high priority throughout the history of human civilization and lies at the origin of agriculture. Specialists and researchers in the fields of agronomy science, agriculture, crop science, horticulture, and forest science are even today constantly striving to find and produce plants with an increased growth potential to feed an increasing world population and to guarantee a supply of reproducible raw materials. The robust level of research in these fields of science indicates the level of importance leaders in every geographic environment and climate around the world place on providing sustainable sources of food, feed, chemicals and energy for the population.

Manipulation of crop performance has been accomplished conventionally for centuries through plant breeding. The breeding process is, however, both time-consuming and labor-intensive. Furthermore, appropriate breeding programs must be specially designed for each relevant plant species.

On the other hand, great progress has been made in using molecular genetics approaches to manipulate plants to provide better crops. Through introduction and expression of recombinant nucleic acid molecules in plants, researchers are now poised to provide the community with plant species tailored to grow more efficiently and produce more product despite unique geographic and/or climatic environments. These new approaches have the additional advantage of not being limited to one plant species, but instead being applicable to multiple different plant species (Zhang et al. (2004) Plant Physiol. 135:615).

Despite this progress, today there continues to be a great need for generally applicable processes that improve forest or agricultural plant growth to suit particular needs depending on specific environmental conditions. To this end, the present invention is directed to advantageously manipulating plant size, organ number, plant growth rate, plant architecture and/or biomass to maximize the benefits of various crops depending on the benefit sought and the particular environment in which the crop must grow, characterized by expression of recombinant DNA molecules in plants. These molecules may be from the plant itself, and simply expressed at a higher or lower level, or the molecules may be from different plant species.

### SUMMARY OF THE INVENTION

The present invention, therefore, relates to isolated nucleic acid molecules and polypeptides and their use in making transgenic plants, plant cells, plant materials or seeds of plants having life cycles, particularly plant size, vegetative growth, plant growth rate, organ number, plant architecture and/or biomass, that are altered with respect to wild-type plants grown under similar or identical conditions.

Unless otherwise defined, all technical and scientific terms used herein have the same meaning as commonly understood by one of ordinary skill in the art to which this invention belongs.

### BRIEF DESCRIPTION OF THE FIGURES

FIG. 1. Amino acid sequence alignment of homologues of Leads 80, 81, 113, 114, ME08328, ME01905, ME01770, ME20023 (Clone 18200) and ME21445, SEQ ID NOS. 95, 97, 91, 83, 89, 85, 87, 93, 81. Conserved regions are enclosed in a box. A consensus sequence is shown below the alignment. The sequences shown in FIG. 1 correspond to the following SEQ ID NOs.:

### CeresClone:486120—SEQ ID NO. 111

gi|50912765—SEQ ID NO. 110

### CeresClone:503296—SEQ ID NO. 112

gi|31431968—SEQ ID NO. 109

### CeresClone:336524—SEQ ID NO. 83

### CeresClone:8607—SEQ ID NO. 97

gi|22331645—SEQ ID NO. 106

### CeresClone:18200—SEQ ID NO. 81

### CeresClone:945972—SEQ ID NO. 103

### CeresClone:519—SEQ ID NO. 87

Lead•clone733804•Taxonomy•4565—SEQ ID NO. 95
gi|78708592—SEQ ID NO. 98

### CeresClone:4734—SEQ ID NO. 85

### CeresGdna:1468218—SEQ ID NO. 101

### CeresClone:653656—SEQ ID NO. 93

### CeresClone:663844—SEQ ID NO. 99

### CeresGdna:1530225—SEQ ID NO. 105

### CeresGdna:1449794—SEQ ID NO. 108

### CeresClone:703180—SEQ ID NO. 102

### CeresClone:560681—SEQ ID NO. 89

### CeresClone:560948—SEQ ID NO. 91

### DETAILED DESCRIPTION OF THE INVENTION

### 1. The Invention

The invention of the present application may be described by, but not necessarily limited to, the following exemplary embodiments.

The present invention discloses novel isolated nucleic acid molecules, nucleic acid molecules that interfere with these nucleic acid molecules, nucleic acid molecules that hybridize to these nucleic acid molecules, and isolated nucleic acid molecules that encode the same protein due to the degeneracy of the DNA code. Additional embodiments of the present application further include the polypeptides encoded by the isolated nucleic acid molecules of the present invention.

More particularly, the nucleic acid molecules of the present invention comprise: (a) a nucleotide sequence encoding an amino acid sequence that is at least 85% identical to any one of Leads 80, 81, 113, 114, ME08328, ME01905, ME01770, ME21445 and ME20023, corresponding to SEQ ID Nos. 94, 96, 90, 82, 88, 84, 86, 92, 80, respectively, (b) a nucleotide sequence that is complementary to any one of the nucleotide sequences according to (a), (c) a nucleotide sequence according to any one of SEQ ID Nos. 94, 96, 90, 82, 88, 84, 86, 92, 80, (d) a nucleotide sequence that is in reverse order of any one of the nucleotide sequences according to (c) when read in the 5′ to 3′ direction, (e) a nucleotide sequence able to interfere with any one of the nucleotide sequences according to (a), (f) a nucleotide sequence able to form a hybridized nucleic acid duplex with the nucleic acid according to any one of paragraphs (a)-(e) at a temperature from about 40° C. to about 48° C. below a melting temperature of the hybridized nucleic acid duplex, and (g) a nucleotide sequence encoding any one of amino acid sequences of Leads 80, 81, 113, 114, ME08328, ME01905, ME01770, ME21445 and ME20023, corresponding to SEQ ID NOS. 95, 97, 91, 83, 89, 85, 87, 93, 81, respectively.

Additional embodiments of the present invention include those polypeptide and nucleic acid molecule sequences disclosed in SEQ ID NOS. 94, 95, 96, 97, 90, 91, 82, 83, 88, 89, 84, 85, 86, 87, 92, 93, 80, 81.

The present invention further embodies a vector comprising a first nucleic acid having a nucleotide sequence encoding a plant transcription and/or translation signal, and a second nucleic acid having a nucleotide sequence according to the isolated nucleic acid molecules of the present invention. More particularly, the first and second nucleic acids may be operably linked. Even more particularly, the second nucleic acid may be endogenous to a first organism, and any other nucleic acid in the vector may be endogenous to a second organism. Most particularly, the first and second organisms may be different species.

In a further embodiment of the present invention, a host cell may comprise an isolated nucleic acid molecule according to the present invention. More particularly, the isolated nucleic acid molecule of the present invention found in the host cell of the present invention may be endogenous to a first organism and may be flanked by nucleotide sequences endogenous to a second organism. Further, the first and second organisms may be different species. Even more particularly, the host cell of the present invention may comprise a vector according to the present invention, which itself comprises nucleic acid molecules according to those of the present invention.

In another embodiment of the present invention, the isolated polypeptides of the present invention may additionally comprise amino acid sequences that are at least 85% identical to any one of Leads 80, 81, 113, 114, ME08328, ME01905, ME01770, ME21445 and ME20023, corresponding to SEQ ID Nos. ID NOS. 95, 97, 91, 83, 89, 85, 87, 93, 81, respectively.

Other embodiments of the present invention include methods of introducing an isolated nucleic acid of the present invention into a host cell. More particularly, an isolated nucleic acid molecule of the present invention may be contacted to a host cell under conditions allowing transport of the isolated nucleic acid into the host cell. Even more particularly, a vector as described in a previous embodiment of the present invention, may be introduced into a host cell by the same method.

Methods of detection are also available as embodiments of the present invention. Particularly, methods for detecting a nucleic acid molecule according to the present invention in a sample. More particularly, the isolated nucleic acid molecule according to the present invention may be contacted with a sample under conditions that permit a comparison of the nucleotide sequence of the isolated nucleic acid molecule with a nucleotide sequence of nucleic acid in the sample. The results of such an analysis may then be considered to determine whether the isolated nucleic acid molecule of the present invention is detectable and therefore present within the sample.

A further embodiment of the present invention comprises a plant, plant cell, plant material or seeds of plants comprising an isolated nucleic acid molecule and/or vector of the present invention. More particularly, the isolated nucleic acid molecule of the present invention may be exogenous to the plant, plant cell, plant material or seed of a plant.

A further embodiment of the present invention includes a plant regenerated from a plant cell or seed according to the present invention. More particularly, the plant, or plants derived from the plant, plant cell, plant material or seeds of a plant of the present invention preferably has increased size (in whole or in part), increased vegetative growth, increased organ number and/or increased biomass (sometimes hereinafter collectively referred to as increased biomass), lethality, sterility or ornamental characteristics as compared to a wild-type plant cultivated under identical conditions. Furthermore, the transgenic plant may comprise a first isolated nucleic acid molecule of the present invention, which encodes a protein involved in modulating growth and phenotype characteristics, and a second isolated nucleic acid molecule which encodes a promoter capable of driving expression in plants, wherein the growth and phenotype modulating component and the promoter are operably linked. More preferably, the first isolated nucleic acid may be mis-expressed in the transgenic plant of the present invention, and the transgenic plant exhibits modulated characteristics as compared to a progenitor plant devoid of the polynucleotide, when the transgenic plant and the progenitor plant are cultivated under identical environmental conditions. In another embodiment of the present invention the modulated growth and phenotype characteristics may be due to the inactivation of a particular sequence, using for example an interfering RNA.

A further embodiment consists of a plant, plant cell, plant material or seed of a plant according to the present invention which comprises an isolated nucleic acid molecule of the present invention, wherein the plant, or plants derived from the plant, plant cell, plant material or seed of a plant, has the modulated growth and phenotype characteristics as compared to a wild-type plant cultivated under identical conditions.

The polynucleotide conferring increased biomass or vigor may be mis-expressed in the transgenic plant of the present invention, and the transgenic plant exhibits an increased biomass or vigor as compared to a progenitor plant devoid of the polynucleotide, when the transgenic plant and the progenitor plant are cultivated under identical environmental conditions. In another embodiment of the present invention increased biomass or vigor phenotype may be due to the inactivation of a particular sequence, using for example an interfering RNA.

Another embodiment consists of a plant, plant cell, plant material or seed of a plant according to the present invention which comprises an isolated nucleic acid molecule of the present invention, wherein the plant, or plants derived from the plant, plant cell, plant material or seed of a plant, has increased biomass or vigor as compared to a wild-type plant cultivated under identical conditions.

Another embodiment of the present invention includes methods of enhancing biomass or vigor in plants. More particularly, these methods comprise transforming a plant with an isolated nucleic acid molecule according to the present invention. Preferably, the method is a method of enhancing biomass or vigor in the transformed plant, whereby the plant is transformed with a nucleic acid molecule encoding the polypeptide of the present invention.

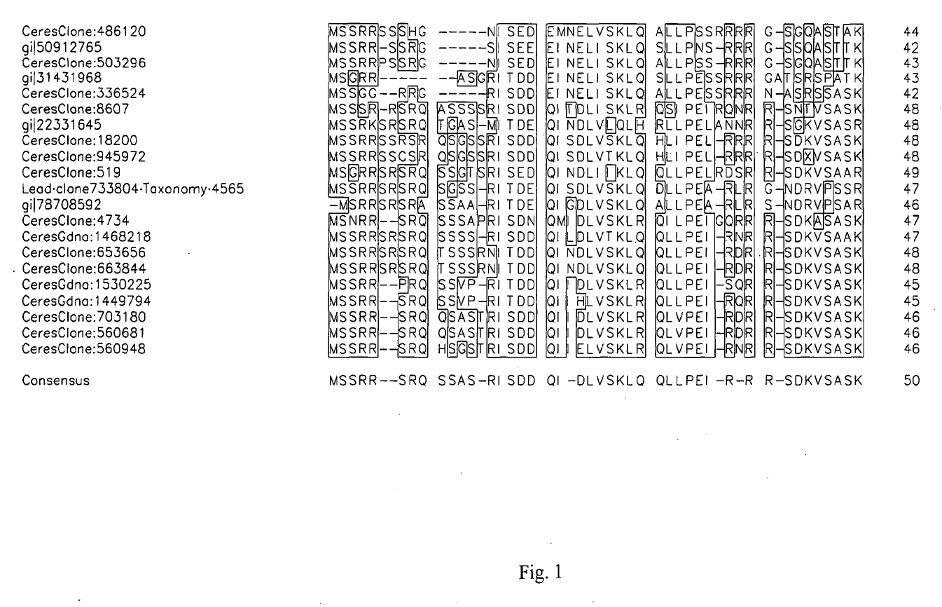

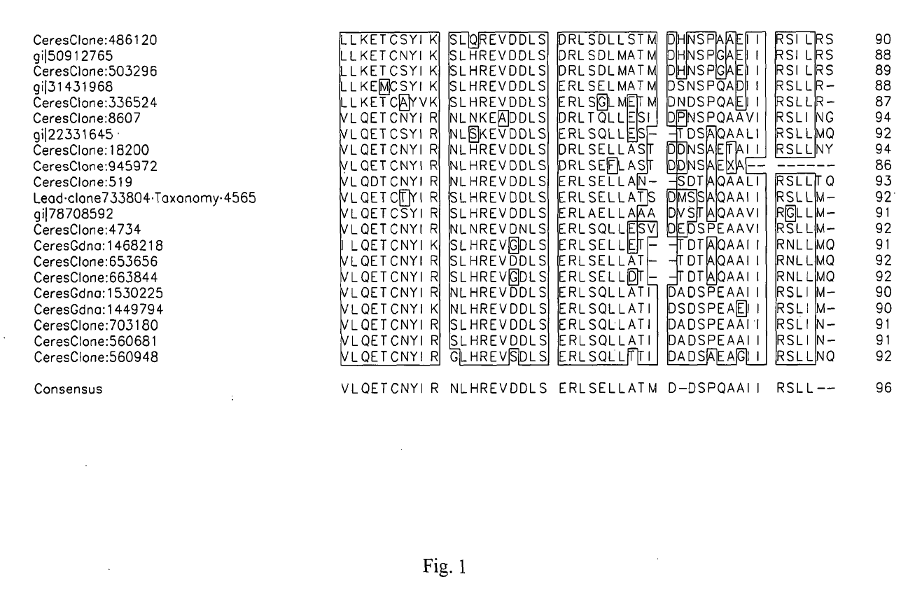

Polypeptides of the present invention include consensus sequences. The consensus sequence is shown in FIG. 1.

### 2. Definitions

The following terms are utilized throughout this application:

Biomass: As used herein, “biomass” refers to useful biological material including a product of interest, which material is to be collected and is intended for further processing to isolate or concentrate the product of interest. “Biomass” may comprise the fruit or parts of it or seeds, leaves, or stems or roots where these are the parts of the plant that are of particular interest for the industrial purpose. “Biomass”, as it refers to plant material, includes any structure or structures of a plant that contain or represent the product of interest.

Transformation: Examples of means by which this can be accomplished are described below and include Agrobacterium-mediated transformation (of dicots (Needleman and Wunsch (1970) J. Mol. Biol. 48:443; Pearson and Lipman (1988) Proc. Natl. Acad. Sci. (USA) 85: 2444), of monocots (Yamauchi et al. (1996) Plant Mol. Biol. 30:321-9; Xu et al. (1995) Plant Mol. Biol. 27:237; Yamamoto et al. (1991) Plant Cell 3:371), and biolistic methods (P. Tijessen, “Hybridization with Nucleic Acid Probes” In Laboratory Techniques in Biochemistry and Molecular Biology, P.C. vand der Vliet, ed., c. 1993 by Elsevier, Amsterdam), electroporation, in planta techniques, and the like. Such a plant containing the exogenous nucleic acid is referred to here as a Tₒ for the primary transgenic plant and T₁ for the first generation.

Functionally Comparable Proteins or Functional Homologs: This term describes those proteins that have at least one functional characteristic in common. Such characteristics include sequence similarity, biochemical activity, transcriptional pattern similarity and phenotypic activity. Typically, the functionally comparable proteins share some sequence similarity or at least one biochemical. Within this definition, analogs are considered to be functionally comparable. In addition, functionally comparable proteins generally share at least one biochemical and/or phenotypic activity.

Functionally comparable proteins will give rise to the same characteristic to a similar, but not necessarily the same, degree. Typically, comparable proteins give the same characteristics where the quantitative measurement due to one of the comparables is at least 20% of the other; more typically, between 30 to 40%; even more typically, between 50-60%; even more typically between 70 to 80%; even more typically between 90 to 100% of the other.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.

Misexpression: The term “misexpression” refers to an increase or a decrease in the transcription of a coding region into a complementary RNA sequence as compared to the wild-type. This term also encompasses expression and/or translation of a gene or coding region or inhibition of such transcription and/or translation for a different time period as compared to the wild-type and/or from a non-natural location within the plant genome, including a gene or coding region from a different plant species or from a non-plant organism.

Percentage of sequence identity: As used herein, the term “percent sequence identity” refers to the degree of identity between any given query sequence and a subject sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). The term “substantial sequence identity” between polynucleotide or polypeptide sequences refers to polynucleotide or polypeptide comprising a sequence that has at least 80% sequence identity, preferably at least 85%, more preferably at least 90% and most preferably at least 95%, even more preferably, at least 96%, 97%, 98% or 99% sequence identity compared to a reference sequence using the programs.

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher website and at the European Bioinformatics Institute website on the World Wide Web.

In case of the functional homolog searches, to ensure a subject sequence having the same function as the query sequence, the alignment has to be along at least 80% of the length of the query sequence so that the majority of the query sequence is covered by the subject sequence. To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

Regulatory Regions: The term “regulatory region” refers to nucleotide sequences that, when operably linked to a sequence, influence transcription initiation or translation initiation or transcription termination of said sequence and the rate of said processes, and/or stability and/or mobility of a transcription or translation product. As used herein, the term “operably linked” refers to positioning of a regulatory region and said sequence to enable said influence. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Regulatory regions can be classified in two categories, promoters and other regulatory regions.

Seedling vigor: As used herein, “seedling vigor” refers to the plant characteristic whereby the plant emerges from soil faster, has an increased germination rate (i.e., germinates faster), has faster and larger seedling growth and/or germinates faster under cold conditions as compared to the wild type or control under similar conditions. Seedling vigor has often been defined to comprise the seed properties that determine “the potential for rapid, uniform emergence and development of normal seedlings under a wide range of field conditions”.

Stringency: “Stringency,” as used herein is a function of nucleic acid molecule probe length, nucleic acid molecule probe composition (G+C content), salt concentration, organic solvent concentration and temperature of hybridization and/or wash conditions. Stringency is typically measured by the parameter Tₘ, which is the temperature at which 50% of the complementary nucleic acid molecules in the hybridization assay are hybridized, in terms of a temperature differential from Tₘ. High stringency conditions are those providing a condition of Tₘ −5° C. to Tₘ −10° C. Medium or moderate stringency conditions are those providing Tₘ −20° C. to Tₘ −29° C. Low stringency conditions are those providing a condition of Tₘ −40° C. to Tₘ −48° C. The relationship between hybridization conditions and Tₘ (in ° C.) is expressed in the mathematical equation:

Tₘ=81.5−16.6(log₁₀[Na⁺])+0.41(% G+C)−(600/N) (I)

where N is the number of nucleotides of the nucleic acid molecule probe. This equation works well for probes 14 to 70 nucleotides in length that are identical to the target sequence. The equation below, for Tₘ of DNA-DNA hybrids, is useful for probes having lengths in the range of 50 to greater than 500 nucleotides, and for conditions that include an organic solvent (formamide):

Tₘ=81.5+16.6 log {[Na⁺]/(1+0.7[Na⁺])}+0.41(% G+C)−500/L0.63(% formamide) (II)

where L represents the number of nucleotides in the probe in the hybrid (21). The Tₘ of Equation II is affected by the nature of the hybrid: for DNA-RNA hybrids, Tₘ is 10-15° C. higher than calculated; for RNA-RNA hybrids, Tₘ is 20-25° C. higher. Because the Tₘ decreases about 1° C. for each 1% decrease in homology when a long probe is used (Frischauf et al. (1983) J. Mol. Biol, 170: 827-842), stringency conditions can be adjusted to favor detection of identical genes or related family members.

Equation II is derived assuming the reaction is at equilibrium. Therefore, hybridizations according to the present invention are most preferably performed under conditions of probe excess and allowing sufficient time to achieve equilibrium. The time required to reach equilibrium can be shortened by using a hybridization buffer that includes a hybridization accelerator such as dextran sulfate or another high volume polymer.

Stringency can be controlled during the hybridization reaction, or after hybridization has occurred, by altering the salt and temperature conditions of the wash solutions. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

T₀: The term “T₀” refers to the whole plant, explant or callus tissue, inoculated with the transformation medium.

T₁: The term T₁ refers to either the progeny of the Tₒ plant, in the case of whole-plant transformation, or the regenerated seedling in the case of explant or callous tissue transformation.

T₂: The term T₂ refers to the progeny of the T₁ plant. T₂ progeny are the result of self-fertilization or cross-pollination of a T₁ plant.

T₃: The term T₃ refers to second generation progeny of the plant that is the direct result of a transformation experiment. T₃ progeny are the result of self-fertilization or cross-pollination of a T₂ plant.

### 3. Important Characteristics of the Polynucleotides and Polypeptides of the Invention

The nucleic acid molecules and polypeptides of the present invention are of interest because when the nucleic acid molecules are mis-expressed (i.e., when expressed at a non-natural location or in an increased or decreased amount relative to wild-type) they produce plants that exhibit modulated biomass, growth rate, or seedling vigor as compared to wild-type plants, as evidenced by the results of various experiments disclosed below. This trait can be used to exploit or maximize plant products. For example, the nucleic acid molecules and polypeptides of the present invention are used to increase the expression of genes that cause the plant to have modulated biomass, growth rate or seedling vigor.

Because the disclosed sequences and methods increase vegetative growth, and growth rate, the disclosed methods can be used to enhance biomass production. For example, plants that grow vegetatively have an increase biomass production, compared to a plant of the same species that is not genetically modified for substantial vegetative growth. Examples of increases in biomass production include increases of at least 5%, at least 20%, or even at least 50%, when compared to an amount of biomass production by a plant of the same species not growing vegetatively.

The life cycle of flowering plants in general can be divided into three growth phases: vegetative, inflorescence, and floral (late inflorescence phase). In the vegetative phase, the shoot apical meristem (SAM) generates leaves that later will ensure the resources necessary to produce fertile offspring. Upon receiving the appropriate environmental and developmental signals the plant switches to floral, or reproductive, growth and the SAM enters the inflorescence phase (I) and gives rise to an inflorescence with flower primordia. During this phase the fate of the SAM and the secondary shoots that arise in the axils of the leaves is determined by a set of meristem identity genes, some of which prevent and some of which promote the development of floral meristems. Once established, the plant enters the late inflorescence phase where the floral organs are produced. If the appropriate environmental and developmental signals the plant switches to floral, or reproductive, growth are disrupted, the plant will not be able to enter reproductive growth, therefore maintaining vegetative growth.

Seed or seedling vigor is an important characteristic that can greatly influence successful growth of a plant, such as crop plants. Adverse environmental conditions, such as dry, wet, cold or hot conditions, can affect a plant growth cycle, and the vigor of seeds (i.e. vitality and strength under such conditions can differentiate between successful and failed crop growth). Seedling vigor has often been defined to comprise the seed properties that determine “the potential for rapid, uniform emergence and development of normal seedlings under a wide range of field conditions”. Hence, it would be advantageous to develop plant seeds with increased vigor.

For example, increased seedling vigor would be advantageous for cereal plants such as rice, maize, wheat, etc. production. For these crops, growth can often be slowed or stopped by cool environmental temperatures during the planting season. In addition, rapid emergence and tillering of rice would permit growers to initiate earlier flood irrigation which can save water and suppress weak growth. Genes associated with increased seed vigor and/or cold tolerance in rice, have therefore been sought for producing improve rice varieties. See e.g., Pinson, S., “Molecular Mapping of Seedling Vigor QTLs in Tropical Rice”, USDA Agricultural Research Service, Dec. 16, 2000.

Seedling vigor has been measured by different tests and assays, including most typically a cold tolerance test and an accelerated aging test.

Some of the nucleotide sequences of the invention code for basic-helix-loop (bHCH) transcription factors. It is known that transcription factors often control the expression of multiple genes in a pathway. The basic/helix-loop-helix (BHLH) proteins are a superfamily of transcription factors that bind as dimers to specific DNA target sites. The bHLH transcription factors have been well characterized in nonplant eukaryotes and have been identified as important regulatory components in diverse biological processes. Many different functions have been identified for those proteins in animals, including the control of cell proliferation and transcription often involves homo- or hetero-dimerization. Members of the R/B basic helix-loop-helix (bHLH) family of plant transcription factors are involved in a variety of growth and differentiation processes.

A basic-helix-loop-helix (bHLH) is a protein structural motif that characterizes a family of transcription factors. The motif is characterized by two a helices connected by a loop. Transcription factors of this type are typically dimeric, each with one helix containing basic amino acid residues that facilitate DNA binding. One helix is typically smaller and due to the flexibility of the loop allows dimerization by folding and packing against another helix. The larger helix typically contains the DNA binding regions. bHLH proteins typically bind to a consensus sequence called an E-box, CANNTG. The canonical E-box is CACGTG, however some bHLH transcription factors bind to different sequences, which are often similar to the E-box. bHLH transcription factors are often important in development or cell activity.

### 4. The Polypeptides/Polynucleotides of the Invention

The polynucleotides of the present invention and the proteins expressed via translation of these polynucleotides are set forth in the Sequence Listing, specifically SEQ ID NOS. 94, 95, 96, 97, 90, 91, 82, 83, 88, 89, 84, 85, 86, 87, 92, 93, 80, 81. The Sequence Listing also consists of functionally comparable proteins. Polypeptides comprised of a sequence within and defined by one of the consensus sequences can be utilized for the purposes of the invention, namely to make transgenic plants with modulated biomass, growth rate and/or seedling vigor.

### 5. Use of the Polypeptides to Make Transgenic Plants

To use the sequences of the present invention or a combination of them or parts and/or mutants and/or fusions and/or variants of them, recombinant DNA constructs are prepared that comprise the polynucleotide sequences of the invention inserted into a vector and that are suitable for transformation of plant cells. The construct can be made using standard recombinant DNA techniques (see, Sambrook et al., Molecular Cloning: A Laboratory Manual, Second Edition, Cold Spring Harbor Laboratory Press, 1989, New York.) and can be introduced into the plant species of interest by, for example, Agrobacterium-mediated transformation, or by other means of transformation, for example, as disclosed below.

The vector backbone may be any of those typically used in the field such as plasmids, viruses, artificial chromosomes, BACs, YACs, PACs and vectors such as, for instance, bacteria-yeast shuttle vectors, lambda phage vectors, T-DNA fusion vectors and plasmid vectors (see, Shizuya et al. (1992) Proc. Natl. Acad. Sci. USA, 89: 8794-8797; Hamilton et al. (1996) Proc. Natl. Acad. Sci. USA, 93: 9975-9979; Burke et al. (1987) Science, 236:806-812; Sternberg N. et al. (1990) Proc Natl Acad Sci USA., 87:103-7; Bradshaw et al. (1995) Nucl Acids Res, 23: 4850-4856; Frischauf et al. (1983) J. Mol. Biol, 170: 827-842; Huynh et al., Glover NM (ed) DNA Cloning: A practical Approach, Vol. 1 Oxford: IRL Press (1985); Walden et al. (1990) Mol Cell Biol 1: 175-194).

Typically, the construct comprises a vector containing a nucleic acid molecule of the present invention with any desired transcriptional and/or translational regulatory sequences such as, for example, promoters, UTRs, and 3′ end termination sequences. Vectors may also include, for example, origins of replication, scaffold attachment regions (SARs), markers, homologous sequences, and introns. The vector may also comprise a marker gene that confers a selectable phenotype on plant cells. The marker may preferably encode a biocide resistance trait, particularly antibiotic resistance, such as resistance to, for example, kanamycin, bleomycin, or hygromycin, or herbicide resistance, such as resistance to, for example, glyphosate, chlorosulfuron or phosphinotricin.

It will be understood that more than one regulatory region may be present in a recombinant polynucleotide, e.g., introns, enhancers, upstream activation regions, transcription terminators, and inducible elements. Thus, more than one regulatory region can be operably linked to said sequence.

To “operably link” a promoter sequence to a sequence, the translation initiation site of the translational reading frame of said sequence is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell 1:977-984 (1989).

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to said sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano, et al., Plant Cell, 1:855-866 (1989); Bustos, et al., Plant Cell, 1:839-854 (1989); Green, et al., EMBO J. 7, 4035-4044 (1988); Meier, et al., Plant Cell, 3, 309-316 (1991); and Zhang, et al., Plant Physiology 110: 1069-1079 (1996).

Examples of various classes of promoters are described below. Some of the promoters indicated below are described in more detail in U.S. Patent Application Ser. Nos. 60/505,689; 60/518,075; 60/544,771; 60/558,869; 60/583,691; 60/619,181; 60/637,140; 10/950,321; 10/957,569; 11/058,689; 11/172,703; 11/208,308; and PCT/US05/23639. It will be appreciated that a promoter may meet criteria for one classification based on its activity in one plant species, and yet meet criteria for a different classification based on its activity in another plant species.

Other Regulatory Regions: A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

Various promoters can be used to drive expression of the polynucleotides of the present invention. Nucleotide sequences of such promoters are set forth in SEQ ID NOS: 1-79. Some of them can be broadly expressing promoters, others may be more tissue preferential.

A promoter can be said to be “broadly expressing” when it promotes transcription in many, but not necessarily all, plant tissues or plant cells. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO: 76), YP0144 (SEQ ID NO: 55), YP0190 (SEQ ID NO: 59), p13879 (SEQ ID NO: 75), YP0050 (SEQ ID NO: 35), p32449 (SEQ ID NO: 77), 21876 (SEQ ID NO: 1), YP0158 (SEQ ID NO: 57), YP0214 (SEQ ID NO: 61), YP0380 (SEQ ID NO: 70), PT0848 (SEQ ID NO: 26), and PT0633 (SEQ ID NO: 7). Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root-active promoters drive transcription in root tissue, e.g., root endodermis, root epidermis, or root vascular tissues. In some embodiments, root-active promoters are root-preferential promoters, i.e., drive transcription only or predominantly in root tissue. Root-preferential promoters include the YP0128 (SEQ ID NO: 52), YP0275 (SEQ ID NO: 63), PT0625 (SEQ ID NO: 6), PT0660 (SEQ ID NO: 9), PT0683 (SEQ ID NO: 14), and PT0758 (SEQ ID NO: 22). Other root-preferential promoters include the PT0613 (SEQ ID NO: 5), PT0672 (SEQ ID NO: 11), PT0688 (SEQ ID NO: 15), and PT0837 (SEQ ID NO: 24), which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA 86:7890-7894 (1989)), root cell specific promoters reported by Conkling et al., Plant Physiol. 93:1203-1211 (1990), and the tobacco RD2 gene promoter.

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin gene promoter (Bustos et al. (1989) Plant Cell 1(9):839-853), the soybean trypsin inhibitor promoter (Riggs et al. (1989) Plant Cell 1(6):609-621), the ACP promoter (Baerson et al. (1993) Plant Mol Biol, 22(2):255-267), the stearoyl-ACP desaturase gene (Slocombe et al. (1994) Plant Physiol 104(4):167-176), the soybean a′ subunit of β-conglycinin promoter (Chen et al. (1986) Proc Natl Acad Sci USA 83:8560-8564), the oleosin promoter (Hong et al. (1997) Plant Mol Biol 34(3):549-555), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al. (1993) Mol. Cell. Biol. 13:5829-5842), the beta-amylase gene promoter, and the barley hordein gene promoter. Other maturing endosperm promoters include the YP0092 (SEQ ID NO: 38), PT0676 (SEQ ID NO: 12), and PT0708 (SEQ ID NO: 17.

Promoters that drive transcription in ovary tissues such as the ovule wall and mesocarp can also be useful, e.g., a polygalacturonidase promoter, the banana TRX promoter, and the melon actin promoter. Other such promoters that drive gene expression preferentially in ovules are YP0007 (SEQ ID NO: 30), YP0111 (SEQ ID NO: 46), YP0092 (SEQ ID NO: 38), YP0103 (SEQ ID NO: 43), YP0028 (SEQ ID NO: 33), YP0121 (SEQ ID NO: 51), YP0008 (SEQ ID NO: 31), YP0039 (SEQ ID NO: 34), YP0115 (SEQ ID NO: 47), YP0119 (SEQ ID NO: 49), YP0120 (SEQ ID NO: 50) and YP0374 (SEQ ID NO: 68).

In some other embodiments of the present invention, embryo sac/early endosperm promoters can be used in order drive transcription of the sequence of interest in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO: 34), YP0101 (SEQ ID NO: 41), YP0102 (SEQ ID NO: 42), YP0110 (SEQ ID NO: 45), YP0117 (SEQ ID NO: 48), YP0119 (SEQ ID NO: 49), YP0137 (SEQ ID NO: 53), DME, YP0285 (SEQ ID NO: 64), and YP0212 (SEQ ID NO: 60). Other promoters that may be useful include the following rice promoters: p530c10, pOsFIE2-2, pOsMEA, pOsYp102, and pOsYp285.

Promoters that preferentially drive transcription in zygotic cells following fertilization can provide embryo-preferential expression and may be useful for the present invention. Most suitable are promoters that preferentially drive transcription in early stage embryos prior to the heart stage, but expression in late stage and maturing embryos is also suitable. Embryo-preferential promoters include the barley lipid transfer protein (Ltpl) promoter (Plant Cell Rep (2001) 20:647-654, YP0097 (SEQ ID NO: 40), YP0107 (SEQ ID NO: 44), YP0088 (SEQ ID NO: 37), YP0143 (SEQ ID NO: 54), YP0156 (SEQ ID NO: 56), PT0650 (SEQ ID NO: 8), PT0695 (SEQ ID NO: 16), PT0723 (SEQ ID NO: 19), PT0838 (SEQ ID NO: 25), PT0879 (SEQ ID NO: 28) and PT0740 (SEQ ID NO: 20).

Promoters active in photosynthetic tissue in order to drive transcription in green tissues such as leaves and stems are of particular interest for the present invention. Most suitable are promoters that drive expression only or predominantly such tissues. Examples of such promoters include the ribulose-1,5-bisphosphate carboxylase (RbcS) promoters such as the RbcS promoter from eastern larch (Larix laricina), the pine cab6 promoter (Yamamoto et al. (1994) Plant Cell Physiol. 35:773-778), the Cab-1 gene promoter from wheat (Fejes et al. (1990) Plant Mol. Biol. 15:921-932), the CAB-1 promoter from spinach (Lubberstedt et al. (1994) Plant Physiol. 104:997-1006), the cab1R promoter from rice (Luan et al. (1992) Plant Cell 4:971-981), the pyruvate orthophosphate dikinase (PPDK) promoter from corn (Matsuoka et al. (1993) Proc Natl Acad. Sci. USA 90:9586-9590), the tobacco Lhcb1*2 promoter (Cerdan et al. (1997) Plant Mol. Biol. 33:245-255), the Arabidopsis thaliana SUC2 sucrose-H+ symporter promoter (Truernit et al. (1995) Planta 196:564-570), and thylakoid membrane protein promoters from spinach (psaD, psaF, psaE, PC, FNR, atpC, atpD, cab, rbcS. Other promoters that drive transcription in stems, leafs and green tissue are PT0535 (SEQ ID NO: 3), PT0668 (SEQ ID NO: 2), PT0886 (SEQ ID NO: 29), PRO924 (SEQ ID NO: 78), YP0144 (SEQ ID NO: 55), YP0380 (SEQ ID NO: 70) and PT0585 (SEQ ID NO: 4).

In some other embodiments of the present invention, inducible promoters may be desired. Inducible promoters drive transcription in response to external stimuli such as chemical agents or environmental stimuli. For example, inducible promoters can confer transcription in response to hormones such as giberellic acid or ethylene, or in response to light or drought. Examples of drought inedible promoters are YP0380 (SEQ ID NO: 70), PT0848 (SEQ ID NO: 26), YP0381 (SEQ ID NO: 71), YP0337 (SEQ ID NO: 66), YP0337 (SEQ ID NO: 66), PT0633 (SEQ ID NO: 7), YP0374 (SEQ ID NO: 68), PT0710 (SEQ ID NO: 18), YP0356 (SEQ ID NO: 67), YP0385 (SEQ ID NO: 73), YP0396 (SEQ ID NO: 74), YP0384 (SEQ ID NO: 72), YP0384 (SEQ ID NO: 72), PT0688 (SEQ ID NO: 15), YP0286 (SEQ ID NO: 65), YP0377 (SEQ ID NO: 69), and PD1367 (SEQ ID NO: 79). Examples of promoters induced by nitrogen are PT0863 (SEQ ID NO: 27), PT0829 (SEQ ID NO: 23), PT0665 (SEQ ID NO: 10) and PT0886 (SEQ ID NO: 29). An example of a shade inducible promoter is PRO924 (SEQ ID NO: 78).

Other Promoters: Other classes of promoters include, but are not limited to, leaf-preferential, stem/shoot-preferential, callus-preferential, guard cell-preferential, such as PT0678 (SEQ ID NO: 13), and senescence-preferential promoters. Promoters designated YP0086 (SEQ ID NO: 36), YP0188 (SEQ ID NO: 58), YP0263 (SEQ ID NO: 62), PT0758 (SEQ ID NO: 22), PT0743 (SEQ ID NO: 21), PT0829 (SEQ ID NO: 23), YP0119 (SEQ ID NO: 49), and YP0096 (SEQ ID NO: 39), as described in the above-referenced patent applications, may also be useful.

Alternatively, misexpression can be accomplished using a two component system, whereby the first component consists of a transgenic plant comprising a transcriptional activator operatively linked to a promoter and the second component consists of a transgenic plant that comprise a nucleic acid molecule of the invention operatively linked to the target-binding sequence/region of the transcriptional activator. The two transgenic plants are crossed and the nucleic acid molecule of the invention is expressed in the progeny of the plant. In another alternative embodiment of the present invention, the misexpression can be accomplished by having the sequences of the two component system transformed in one transgenic plant line.

Another alternative consists in inhibiting expression of a biomass or vigor-modulating polypeptide in a plant species of interest. The term “expression” refers to the process of converting genetic information encoded in a polynucleotide into RNA through transcription of the polynucleotide (i.e., via the enzymatic action of an RNA polymerase), and into protein, through translation of mRNA. “Up-regulation” or “activation” refers to regulation that increases the production of expression products relative to basal or native states, while “down-regulation” or “repression” refers to regulation that decreases production relative to basal or native states.

A number of nucleic-acid based methods, including anti-sense RNA, ribozyme directed RNA cleavage, and interfering RNA (RNAi) can be used to inhibit protein expression in plants. Antisense technology is one well-known method. In this method, a nucleic acid segment from the endogenous gene is cloned and operably linked to a promoter so that the antisense strand of RNA is transcribed. The recombinant vector is then transformed into plants, as described above, and the antisense strand of RNA is produced. The nucleic acid segment need not be the entire sequence of the endogenous gene to be repressed, but typically will be substantially identical to at least a portion of the endogenous gene to be repressed. Generally, higher homology can be used to compensate for the use of a shorter sequence. Typically, a sequence of at least 30 nucleotides is used (e.g., at least 40, 50, 80, 100, 200, 500 nucleotides or more).

Thus, for example, an isolated nucleic acid provided herein can be an antisense nucleic acid to one of the aforementioned nucleic acids encoding a biomass-modulating polypeptide. A nucleic acid that decreases the level of a transcription or translation product of a gene encoding a biomass-modulating polypeptide is transcribed into an antisense nucleic acid similar or identical to the sense coding sequence of the biomass- or growth rate-modulating polypeptide. Alternatively, the transcription product of an isolated nucleic acid can be similar or identical to the sense coding sequence of a biomass growth rate-modulating polypeptide, but is an RNA that is unpolyadenylated, lacks a 5′ cap structure, or contains an unsplicable intron.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman, et al. (1995) Proc. Natl. Acad. Sci. USA, 92(13):6175-6179; de Feyter and Gaudron, Methods in Molecular Biology, Vol. 74, Chapter 43, “Expressing Ribozymes in Plants”, Edited by Turner, P.C, Humana Press Inc., Totowa, N.J. RNA endoribonucleases such as the one that occurs naturally in Tetrahymena thermophila, and which have been described extensively by Cech and collaborators can be useful. See, for example, U.S. Pat. No. 4,987,071.

Methods based on RNA interference (RNAi) can be used. RNA interference is a cellular mechanism to regulate the expression of genes and the replication of viruses. This mechanism is thought to be mediated by double-stranded small interfering RNA molecules. A cell responds to such a double-stranded RNA by destroying endogenous mRNA having the same sequence as the double-stranded RNA. Methods for designing and preparing interfering RNAs are known to those of skill in the art; see, e.g., WO 99/32619 and WO 01/75164. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises an antisense sequence of the biomass-modulating polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. See, e.g., WO 99/53050.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., 1996, Bioorgan. Med. Chem., 4: 5-23. In addition, the deoxyphosphate backbone can be replaced with, for example, a phosphorothioate or phosphorodithioate backbone, a phosphoroamidite, or an alkyl phosphotries ter backbone.

### Transformation

Nucleic acid molecules of the present invention may be introduced into the genome or the cell of the appropriate host plant by a variety of techniques. These techniques, able to transform a wide variety of higher plant species, are well known and described in the technical and scientific literature (see, e.g., Weising et al. (1988) Ann. Rev. Genet., 22:421 and Christou (1995) Euphytica, 85:13-27).

A variety of techniques known in the art are available for the introduction of DNA into a plant host cell. These techniques include transformation of plant cells by injection (Newell (2000)), microinjection (Griesbach (1987) Plant Sci. 50:69-77), electroporation of DNA (Fromm et al. (1985) Proc. Natl. Acad. Sci. USA 82:5824), PEG (Paszkowski et al. (1984) EMBO J. 3:2717), use of biolistics (Klein et al. (1987) Nature 327:773), fusion of cells or protoplasts (Willmitzer, L. (1993) Transgenic Plants. In: Iotechnology, A Multi-Volume Comprehensive treatise (H.J. Rehm, G. Reed, A. Püler, P. Stadler, eds., Vol. 2, 627-659, VCH Weinheim-New York-Base1-Cambridge), and via T-DNA using Agrobacterium tumefaciens (Crit. Rev. Plant. Sci. 4:1-46; Fromm et al. (1990) Biotechnology 8:833-844) or Agrobacterium rhizogenes (Cho et al. (2000) Planta 210:195-204) or other bacterial hosts (Brootghaerts et al. (2005) Nature 433:629-633), for example.

In addition, a number of non-stable transformation methods that are well known to those skilled in the art may be desirable for the present invention. Such methods include, but are not limited to, transient expression (Lincoln et al. (1998) Plant Mol. Biol. Rep. 16:1-4) and viral transfection (Lacomme et al. (2001), “Genetically Engineered Viruses” (C.J.A. Ring and E.D. Blair, Eds). Pp. 59-99, BIOS Scientific Publishers, Ltd. Oxford, UK).

Seeds are obtained from the transformed plants and used for testing stability and inheritance. Generally, two or more generations are cultivated to ensure that the phenotypic feature is stably maintained and transmitted.

A person of ordinary skill in the art recognizes that after the expression cassette is stably incorporated in transgenic plants and confirmed to be operable, it can be introduced into other plants by sexual crossing. Any of a number of standard breeding techniques can be used, depending upon the species to be crossed.

The nucleic acid molecules of the present invention may be used to confer the trait of an altered flowering time.

The nucleic acid molecules of the present invention encode appropriate proteins from any organism, but are preferably found in plants, fungi, bacteria or animals.

The methods according to the present invention can be applied to any plant, preferably higher plants, pertaining to the classes of Angiospermae and Gymnospermae. Plants of the subclasses of the Dicotylodenae and the Monocotyledonae are particularly suitable. Dicotyledonous plants belonging to the orders of the Magniolales, Illiciales, Laurales, Piperales Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales, for example, are also suitable. Monocotyledonous plants belonging to the orders of the Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales also may be useful in embodiments of the present invention. Further examples include, but are not limited to, plants belonging to the class of the Gymnospermae are Pinales, Ginkgoales, Cycadales and Gnetales.

The methods of the present invention are preferably used in plants that are important or interesting for agriculture, horticulture, biomass for bioconversion and/or forestry. Non-limiting examples include, for instance, tobacco, oilseed rape, sugar beet, potatoes, tomatoes, cucumbers, peppers, beans, peas, citrus fruits, avocados, peaches, apples, pears, berries, plumbs, melons, eggplants, cotton, soybean, sunflowers, roses, poinsettia, petunia, guayule, cabbages, spinach, alfalfa, artichokes, sugarcane, mimosa, Servicea lespedera, corn, wheat, rice, rye, barley, sorghum and grasses such as switch grass, giant reed, Bermuda grass, Johnson grasses or turf grass, millet, hemp, bananas, poplars, eucalyptus trees and conifers. Of interest are plates grown for energy production, so called energy crops, such as broadleaf plants like alfalfa, hemp, Jerusalem artichoke and grasses such as sorgum, switchgrass, Johnson grass and the likes.

### Homologues Encompassed by the Invention

It is known in the art that one or more amino acids in a sequence can be substituted with other amino acid(s), the charge and polarity of which are similar to that of the substituted amino acid, i.e. a conservative amino acid substitution, resulting in a biologically/functionally silent change. Conservative substitutes for an amino acid within the polypeptide sequence can be selected from other members of the class to which the amino acid belongs. Amino acids can be divided into the following four groups: (1) acidic (negatively charged) amino acids, such as aspartic acid and glutamic acid; (2) basic (positively charged) amino acids, such as arginine, histidine, and lysine; (3) neutral polar amino acids, such as serine, threonine, tyrosine, asparagine, and glutamine; and (4) neutral nonpolar (hydrophobic) amino acids such as glycine, alanine, leucine, isoleucine, valine, proline, phenylalanine, tryptophan, cysteine, and methionine.

Nucleic acid molecules of the present invention can comprise sequences that differ from those encoding a protein or fragment thereof selected from the group consisting of Leads 80, 81, 113, 114, ME08328, ME01905, ME01770, ME21445 and ME20023, SEQ ID NOS. 95, 97, 91, 83, 89, 85, 87, 93, and 81, respectively, due to the fact that the different nucleic acid sequence encodes a protein having one or more conservative amino acid changes.

Biologically functional equivalents of the polypeptides, or fragments thereof, of the present invention can have about 10 or fewer conservative amino acid changes, more preferably about 7 or fewer conservative amino acid changes, and most preferably about 5 or fewer conservative amino acid changes. In a preferred embodiment of the present invention, the polypeptide has between about 5 and about 500 conservative changes, more preferably between about 10 and about 300 conservative changes, even more preferably between about 25 and about 150 conservative changes, and most preferably between about 5 and about 25 conservative changes or between 1 and about 5 conservative changes.

### Identification of Useful Nucleic Acid Molecules and Their Corresponding Nucleotide Sequences

The nucleic acid molecules, and nucleotide sequences thereof, of the present invention were identified by use of a variety of screens that are predictive of nucleotide sequences that provide plants with altered size, vegetative growth, growth rate, organ number, plant architecture and/or biomass. One or more of the following screens were, therefore, utilized to identify the nucleotide (and amino acid) sequences of the present invention.

The present invention is further exemplified by the following examples. The examples are not intended to in any way limit the scope of the present application and its uses.

### 6. Experiments Confirming the Usefulness of the Polynucleotides and Polypeptides of the Invention

### General Protocols

### Agrobacterium-Mediated Transformation of Arabidopsis

Wild-type Arabidopsis thaliana Wassilewskija (WS) plants are transformed with Ti plasmids containing clones in the sense orientation relative to the 35S promoter. A Ti plasmid vector useful for these constructs, CRS 338, contains the Ceres-constructed, plant selectable marker gene phosphinothricin acetyltransferase (PAT), which confers herbicide resistance to transformed plants.

Ten independently transformed events are typically selected and evaluated for their qualitative phenotype in the T₁ generation.

Preparation of Soil Mixture: 24L SunshineMix #5 soil (Sun Gro Horticulture, Ltd., Bellevue, Wash.) is mixed with 16L Therm-O-Rock vermiculite (Therm-O-Rock West, Inc., Chandler, Ariz.) in a cement mixer to make a 60:40 soil mixture. To the soil mixture is added 2 Tbsp Marathon 1% granules (Hummert, Earth City, Mo.), 3 Tbsp OSMOCOTE® 14-14-14 (Hummert, Earth City, Mo.) and 1 Tbsp Peters fertilizer 20-20-20 (J.R. Peters, Inc., Allentown, Pa.), which are first added to 3 gallons of water and then added to the soil and mixed thoroughly. Generally, 4-inch diameter pots are filled with soil mixture. Pots are then covered with 8-inch squares of nylon netting.

Planting: Using a 60 mL syringe, 35 mL of the seed mixture is aspirated. 25 drops are added to each pot. Clear propagation domes are placed on top of the pots that are then placed under 55% shade cloth and subirrigated by adding 1 inch of water.

Plant Maintenance: 3 to 4 days after planting, lids and shade cloth are removed. Plants are watered as needed. After 7-10 days, pots are thinned to 20 plants per pot using forceps. After 2 weeks, all plants are subirrigated with Peters fertilizer at a rate of 1 Tsp per gallon of water. When bolts are about 5-10 cm long, they are clipped between the first node and the base of stem to induce secondary bolts. Dipping infiltration is performed 6 to 7 days after clipping.

Preparation of Agrobacterium: To 150 mL fresh YEB is added 0.1 mL each of carbenicillin, spectinomycin and rifampicin (each at 100 mg/ml stock concentration). Agrobacterium starter blocks are obtained (96-well block with Agrobacterium cultures grown to an OD₆₀₀ of approximately 1.0) and inoculated one culture vessel per construct by transferring 1 mL from appropriate well in the starter block. Cultures are then incubated with shaking at 27° C. Cultures are spun down after attaining an 0D₆₀₀ of approximately 1.0 (about 24 hours). 200 mL infiltration media is added to resuspend Agrobacterium pellets. Infiltration media is prepared by adding 2.2 g MS salts, 50 g sucrose, and 5 μl 2 mg/ml benzylaminopurine to 900 ml water.

Dipping Infiltration: The pots are inverted and submerged for 5 minutes so that the aerial portion of the plant is in the Agrobacterium suspension. Plants are allowed to grow normally and seed is collected.

High-throughput Phenotypic Screening of Misexpression Mutants: Seed is evenly dispersed into water-saturated soil in pots and placed into a dark 4° C. cooler for two nights to promote uniform germination. Pots are then removed from the cooler and covered with 55% shade cloth for 4-5 days. Cotyledons are fully expanded at this stage. FINALE® (Sanofi Aventis, Paris, France) is sprayed on plants (3 ml FINALE® diluted into 48 oz. water) and repeated every 3-4 days until only transformants remain.

Screening: Screening is routinely performed at four stages: Seedling, Rosette, Flowering, and Senescence.
Seedling—the time after the cotyledons have emerged, but before the 3^{rd} true leaf begins to form.
Rosette—the time from the emergence of the 3^{rd} true leaf through just before the primary bolt begins to elongate.
Flowering—the time from the emergence of the primary bolt to the onset of senescence (with the exception of noting the flowering time itself, most observations should be made at the stage where approximately 50% of the flowers have opened).
Senescence—the time following the onset of senescence (with the exception of “delayed senescence”, most observations should be made after the plant has completely dried). Seeds are then collected.

Screens: Screening for increased size, vegetative growth and/or biomass is performed by taking measurements, specifically T₂ measurements were taken as follows:
Days to Bolt=number of days between sowing of seed and emergence of first inflorescence.
Rosette Leaf Number at Bolt=number of rosette leaves present at time of emergence of first inflorescence.
Rosette Area=area of rosette at time of initial inflorescence emergence, using formula ((L×W)*3.14)/4.
Height=length of longest inflorescence from base to apex. This measurement was taken at the termination of flowering/onset of senescence.
Primary Inflorescence Thickness=diameter of primary inflorescence 2.5 cm up from base. This measurement was taken at the termination of flowering/onset of senescence.
Inflorescence Number=total number of unique inflorescences. This measurement was taken at the termination of flowering/onset of senescence.

PCR was used to amplify the cDNA insert in one randomly chosen T₂ plant. This PCR product was then sequenced to confirm the sequence in the plants.

Screening Superpools for Tolerance to Low Ammonium Nitrate Growth Conditions:

Superpools are generated and two thousand seeds each from ten superpools are pooled together and assayed using the Low Ammonium Nitrate Screen on Agar. Low ammonium nitrate growth media, pH 5.7, is as follows: 0.5×MS without N (PhytoTech), 0.5% sucrose (Sigma), 240 μM NH₄NO₃ (EM Science), 0.5 g MES hydrate (Sigma), 0.8% Phytagar (EM Science). Forty-five (45) ml of media per square plate is used.

Arabidopsis thaliana cv WS seed is sterilized in 50% Clorox™ with 0.01% Triton X-100 (v/v) for five minutes, washed four times with sterile distilled deionized water and stored at 4° C. in the dark for 3 days prior to use.

Seed is plated at a density of 100 seeds per plate. Wild-type seed is used as a control. Plates are incubated in a Conviron™ growth chamber at 22° C. with a 16:8 hour light:dark cycle from a combination of incandescent and fluorescent lamps emitting a light intensity of ˜100 μEinsteins and 70% humidity.

Seedlings are screened daily after 14 days. Candidate seedlings are larger or stay greener longer relative to wild-type controls. DNA is isolated from each candidate plant and sequenced to determine which transgene was present.

Seedling Low Ammonium Nitrate Assay On Agar:

Media and seeds are prepared as described above.

Seeds from five misexpression line events, each containing the same polynucleotide, are sown in two rows, with ten seeds per row. Each plate contains five events, for a total of 100 seeds. Control plates containing wild-type seed are also prepared. Plates are then incubated at 4° C. for at least two days.

After the several day 4° C. cold treatment, plates are incubated in a Conviron™ growth chamber at 22° C. with a 16:8 hour light: dark cycle from a combination of incandescent and fluorescent lamps emitting a light intensity of ˜100 μEinsteins and 70% humidity.

After 14 days, plates are scanned daily using a CF Imager (Technologica Ltd.) with a 45 minute dark acclimation. The CF Imager is used to quantify the seedlings' optimum quantum yields (Fv/Fm) as a measure of photosynthetic health (see details below). To quantify the seedlings' sizes, plates are also scanned with a flatbed photo scanner (Epson) one day after nitrogen stress is apparent and wild-type seedling growth is arrested. Image capture is ended after all wild-type plants have completely yellowed. On the final scanning day plates are uncovered and liberally sprayed with Finale™ (10 ml in 48 oz. Murashige & Skoog liquid media) and returned to the growth chamber.

Two days after spraying, the plates are placed in a closed box for 45 minutes to acclimate in preparation for fluorescence visualization via CF Imager. Plants resistant to Finale™ appear red while sensitive plants appear blue. After image capture, plants are assigned a transgenic (resistant) or non-transgenic (sensitive) status. The non-transgenic plants (i.e. non-transgenic segregants) serve as internal controls.

Seedling photosynthetic efficiency, or electron transport via photosystem II, is estimated by the relationship between Fm, the maximum fluorescence signal and the variable fluorescence, Fv. Here, a reduction in the optimum quantum yield (Fv/Fm) indicates stress, and so can be used to monitor the performance of transgenic plants compared to non-transgenic plants under nitrogen stress conditions. Since a large amount of nitrogen is invested in maintaining the photosynthetic apparatus, nitrogen deficiencies can lead to dismantling of the reaction centers and to reductions in photosynthetic efficiency. Consequently, from the start of image capture collection until the plants are dead the Fv/Fm ratio is determined for each seedling using the Fluorolmager 2 software (Kevin Oxborough and John Bartington).

The rosette area of each plant is also analyzed using WinRHIZO software (Regent Instruments) to analyze the Epson flatbed scanner captured images.

Low Ammonium Nitrate Validated Assay:

Media and seeds are prepared as described above.

For misexpression lines which pass the above low ammonium nitrate assay, both T₂ and T₃ generation seed for an event are plated along with wild-type seed, at a final density of 100 seeds per plate. Plates contain 10 seed/row and have four rows of 10 T₂ seed followed by two rows of wild-type seed, followed by four rows of T₃ seed. Plates are then incubated at 4° C. for at least two days.

After the several day 4° C. cold treatment, plates are incubated in a Conviron™ growth chamber at 22° C. with a 16:8 hour light:dark cycle from a combination of incandescent and fluorescent lamps emitting a light intensity of ˜100 μEinsteins and 70% humidity.

After 14 days, plates are scanned daily using a CF Imager (Technologica Ltd.) with a 45 minute dark acclimation. The CF Imager is used to quantify the seedlings' optimum quantum yields (Fv/Fm) as a measure of photosynthetic health. To quantify the seedlings' sizes, plates are also scanned with a flatbed photo scanner (Epson) one day after nitrogen stress is apparent and wild-type seedling growth is arrested. Image capture is ended after all wild-type plants have completely yellowed. On the final scanning day plates are uncovered and liberally sprayed with Finale™ (10 ml in 48 oz. Murashige & Skoog liquid media) and returned to the growth chamber.

Two days after spraying, the plates are placed in a closed box for 45 minutes to acclimate in preparation for fluorescence visualization via CF Imager. Plants resistant to Finale™ appear red while sensitive plants appear blue. After image capture, plants are assigned a transgenic (resistant) or non-transgenic (sensitive) status. The non-transgenic plants (i.e. non-transgenic segregants) serve as internal controls.

Fv/Fm ratio is determined for each seedling using the Fluorolmager 2 software (Kevin Oxborough and John Bartington).

The rosette area of each plant is also analyzed using WinRHIZO software (Regent Instruments) to analyze the Epson flatbed scanner captured images.

### Results:

Plants transformed with the genes of interest were screened as described above for modulated growth and phenotype characteristics. The observations include those with respect to the entire plant, as well as parts of the plant, such as the roots and leaves. The observations for transformants with each polynucleotide sequence are noted in the Sequence listing for each of the tested nucleotide sequences and the corresponding encoded polypeptide. The modulated characteristics (i.e. observed phenotypes) are noted by an entry in the “miscellaneous features” field for each respective sequence. The “Phenotype” noted in the Sequence Listing for each relevant sequence further includes a statement of the useful utility of that sequence based on the observations.

The observations made for the various transformants can be categorized, depending upon the relevant plant tissue for the observation and the consequent utility/usefulness of the nucleotide sequence/polypeptide used to make that transformant. Table 1 correlates the shorthand notes in the sequence listing to the observations noted for each transformant (the “description” column), the tissue of the observation, the phenotype thereby associated with the transformant, and the consequent utility/usefulness of the inserted nucleotide sequence and encoded polypeptide (the “translation” column).

For some of the polynucleotides/polypeptides of the invention, the sequence listing further includes (in a “miscellaneous feature” section) an indication of important identified dominant(s) and the corresponding function of the domain or identified by comparison to the publicly available pfam database.

TABLE 1

| | PHENOTYPE | | | |
|---|---|---|---|---|
| TISSUE | QUALIFIER | PHENOTYPE | DESCRIPTION | TRANSLATION |
| WHOLE | Senescence Time | Early | the plant senesces | Useful for accelerating |
| PLANT | | Senescence | significantly early | crop development and |
| | | | (note the approximate | harvest |
| | | | number of days early | |
| | | | it started to senesce | |
| | | | in the comments) | |
| INFLORESCENCE | Flowering Time | Early Flowering | the plant flowers | Useful for accelerating |
| | | | significantly early | flowering time |
| | | | (note the approximate | |
| | | | number of days early | |
| | | | it flowered in the | |
| | | | comments) | |
| INFLORESCENCE | Flowering Time | Late Flowering | the plant flowers | Useful for delaying |
| | | | significantly late | flowering time |
| | | | (note the approximate | |
| | | | number of days late it | |
| | | | flowered in the | |
| | | | comments) | |
| INFLORESCENCE | Flowering Time | Dtb | days to bolt | Useful for delaying |
| | | | | flowering time |
| WHOLE | Senescence Time | Late Senescence | the plant senesces | Useful for delaying |
| PLANT | | | significantly late | senescence |
| | | | (note the approximate | |
| | | | number of days late it | |
| | | | started to senesce in | |
| | | | the comments) | |
| COTYLEDONS | Silver | Silver | cotyledons have a | Useful for drought or |
| | | | gray/silver colored | stress tolerance |
| | | | surface; This | |
| | | | phenotype is often | |
| | | | accompanied by a | |
| | | | small size mutation, | |
| | | | but not always | |
| WHOLE | Dark Green | Dark Green | plant is visibly darker | Useful for increasing |
| SEEDLING | | | green | chlorophyll and |
| | | | | photosynthetic capacity |
| WHOLE | Color | Dark Green | the plant is | Useful for increasing |
| PLANT | | | abnormally dark | chlorophyll and |
| | | | green | photosynthetic capacity |
| WHOLE | High | High | the plant is purple in | Useful for increasing |
| SEEDLING | Anthocyanin | Anthocyanin | color | increasing anthocyanin |
| | | | | content |
| WHOLE | Color | High | the plant is purple in | Useful for increasing |
| PLANT | | Anthocyanin | color | increasing anthocyanin |
| | | | | content |
| ROOT | No Growth in | No Growth in | roots grow along the | Useful for increasing root |
| | Soil | Soil | soil surface instead of | growth eg to enhance |
| | | | into the soil | nutrient uptake |
| ROOT | Other | Other | this correlates with | Useful for increasing root |
| | | | any root mutant | growth eg to enhance |
| | | | phenotypes which do | nutrient uptake |
| | | | not fit into the above | |
| | | | categories (a picture | |
| | | | should be taken for | |
| | | | documentation) | |
| LATERAL | Number | Less Lateral | there is an | Useful for increasing root |
| ROOTS | | Roots | abnormally low | growth eg to enhance |
| | | | number of lateral | nutrient uptake |
| | | | roots | |
| LATERAL | Other | Other | this correlates with | Useful for increasing root |
| ROOTS | | | any lateral root | growth eg to enhance |
| | | | mutant phenotypes | nutrient uptake |
| | | | which do not fit into | |
| | | | the above categories | |
| | | | (a picture should be | |
| | | | taken for | |
| | | | documentation) | |
| ROOT | Classic | Classic | there is a lack of | Useful for increasing root |
| | | | lateral roots (buds | growth eg to enhance |
| | | | may appear but do | nutrient uptake |
| | | | not elongate) | |
| ROOT | Dwarf | Dwarf | there is a stunted root | Useful for increasing root |
| | | | system | growth eg to enhance |
| | | | | nutrient uptake |
| ROOT | Mid-Section | Mid-Section | there are lateral roots | Useful for increasing root |
| | | | in the top and bottom | growth eg to enhance |
| | | | quarters of the whole | nutrient uptake |
| | | | root, but none in the | |
| | | | middle | |
| ROOT | Split | Split | appears as “classic” | Useful for increasing root |
| | | | but with two primary | growth eg to enhance |
| | | | roots, both | nutrient uptake |
| | | | originating from the | |
| | | | hypocotyl base | |
| ROOT | Other | Other | this correlates with | Useful for increasing root |
| | | | any overall root | growth eg to enhance |
| | | | structure mutant | nutrient uptake |
| | | | phenotypes which do | |
| | | | not fit into the above | |
| | | | categories (a picture | |
| | | | should be taken for | |
| | | | documentation) | |
| PRIMARY | Other | Other | this correlates with | Useful for increasing root |
| ROOT | | | any primary root | growth eg to enhance |
| | | | mutant phenotypes | nutrient uptake |
| | | | which do not fit into | |
| | | | the above categories | |
| | | | (a picture should be | |
| | | | taken for | |
| | | | documentation) | |
| ROOT | Length | Longer Root | the root hairs are | Useful for increasing root |
| HAIRS | | Hair | abnormally long | growth eg to enhance |
| | | | | nutrient uptake |
| ROOT | Length | Smaller Root | the root hairs are | Useful for increasing root |
| HAIRS | | Hair | abnormally short | growth eg to enhance |
| | | | | nutrient uptake |
| ROOT | Number | Less root hairs | there is an | Useful for increasing root |
| HAIRS | | | abnormally low | growth eg to enhance |
| | | | number of root hairs | nutrient uptake |
| ROOT | Other | Other | this correlates with | Useful for increasing root |
| HAIRS | | | any root hair mutant | growth eg to enhance |
| | | | phenotypes which do | nutrient uptake |
| | | | not fit into the above | |
| | | | categories (a picture | |
| | | | should be taken for | |
| | | | documentation) | |
| ROOT | Bulbous Root | Bulbous Root | Bulbous Root Hairs | Useful for increasing root |
| HAIRS | Hairs | Hairs | | growth eg to enhance |
| | | | | nutrient uptake |
| ROOT | Bearded | Bearded | the lateral roots are | Useful for increasing root |
| | (Nitrogen) | (Nitrogen) | long in high nitrogen, | growth eg to enhance |
| | | | and they are short in | nutrient uptake |
| | | | low nitrogen | |
| PRIMARY | Thickness | Thicker Primary | the primary root is | Useful for increasing root |
| ROOT | | Root | abnormally thick | growth eg to enhance |
| | | | | nutrient uptake |
| WHOLE | Stress | Root | Identify plants with | Useful for increasing root |
| PLANT | | Architecture | increased root mass | growth eg to enhance |
| | | | | nutrient uptake |
| PRIMARY | Thickness | Thinner Primary | the primary root is | Useful for increasing root |
| ROOT | | Root | abnormally thin | growth eg to enhance |
| | | | | nutrient uptake |
| PRIMARY | Wavy | Wavy | there is a consistent | Useful for increasing root |
| ROOT | | | and gentle wavy | growth eg to enhance |
| | | | appearance | nutrient uptake |
| LATERAL | Length | Longer Lateral | the lateral roots are | Useful for increasing root |
| ROOTS | | Root | abnormally long | growth eg to enhance |
| | | | | nutrient uptake |
| LATERAL | Number | More Lateral | there is an | Useful for increasing root |
| ROOTS | | Roots | abnormally high | growth eg to enhance |
| | | | number of lateral | nutrient uptake |
| | | | roots | |
| ROOT | Number | More root hairs | there is an | Useful for increasing root |
| HAIRS | | | abnormally high | growth eg to enhance |
| | | | number of root hairs | nutrient uptake |
| | | | | Useful for increasing seed |
| | | | | carbon or nitrogen |
| SEED | Seed Weight | Weight | weight of seed | Useful for increasing seed |
| | | | | weight |
| SILIQUES | Length | Long | siliques are | Useful for increasing |
| | | | abnormally long (the | seed/fruit yield or |
| | | | percent difference in | modifying fruit content |
| | | | length compared to | |
| | | | the control should be | |
| | | | noted in the | |
| | | | comments) | |
| SILIQUES | Length | Short | siliques are | Useful for increasing |
| | | | abnormally short | seed/fruit yield or |
| | | | (the percent | modifying fruit content |
| | | | difference in length | |
| | | | compared to the | |
| | | | control should be | |
| | | | noted in the | |
| | | | comments) | |
| SILIQUES | Other | Other | this correlates with | Useful for increasing |
| | | | any silique mutant | seed/fruit yield or |
| | | | phenotypes which do | modifying fruit content |
| | | | not fit into the above | |
| | | | categories (a picture | |
| | | | should be taken for | |
| | | | documentation) | |
| ROSETTE | Size | Large | rosette leaves are | Useful for increasing |
| LEAVES | | | abnormally large | vegetative growth and |
| | | | (the percent | enhancing foliage |
| | | | difference in size | |
| | | | compared to the | |
| | | | control should be | |
| | | | noted in the | |
| | | | comments) | |
| | | | | Useful for making |
| | | | | nutraceuticals/pharmaceuticals |
| | | | | in plants |
| HYPOCOTYL | Other | Other | this correlates with | Useful for making larger |
| | | | any hypocotyl mutant | plants |
| | | | phenotypes which do | |
| | | | not fit into the above | |
| | | | categories (a picture | |
| | | | should be taken for | |
| | | | documentation) | |
| WHOLE | Other | Other | this correlates with | Useful for making larger |
| SEEDLING | | | any whole plant | plants |
| | | | mutant phenotypes | |
| | | | which do not fit into | |
| | | | the above categories | |
| | | | (a picture should be | |
| | | | taken for | |
| | | | documentation) | |
| WHOLE | Other | Other | this correlates with | Useful for making larger |
| PLANT | | | any whole plant | plants |
| | | | mutant phenotypes | |
| | | | which do not fit into | |
| | | | the above categories | |
| | | | (a picture should be | |
| | | | taken for | |
| | | | documentation) | |
| CAULINE | Petiole Length | Long Petioles | the cauline petioles | Useful for making larger |
| LEAVES | | | are abnormally long | plants |
| | | | (the percent | |
| | | | difference in size | |
| | | | compared to the | |
| | | | control should be | |
| | | | noted in the | |
| | | | comments) | |
| WHOLE | Size | Large | plant is abnormally | Useful for making larger |
| SEEDLING | | | large (the percent | plants |
| | | | difference in size | |
| | | | compared to the | |
| | | | control should be | |
| | | | noted in the | |
| | | | comments) | |
| WHOLE | Size | Large | plant is abnormally | Useful for making larger |
| PLANT | | | large (the percent | plants |
| | | | difference in size | |
| | | | compared to the | |
| | | | control should be | |
| | | | noted in the | |
| | | | comments) | |
| SEED | Lethal | Lethal | the seed is inviable | Useful for making lethal |
| | | | and appears as a | plants for genetic |
| | | | small, dark, raisin- | confinement systems |
| | | | like seed in the | |
| | | | mature siligue | |
| WHOLE | Germination | No Germination | none of the seed | Useful for making lethal |
| SEEDLING | | | germinates | plants for genetic |
| | | | | confinement systems |
| WHOLE | Germination | Poor | a portion of the seed | Useful for making lethal |
| SEEDLING | | Germination | never germinates | plants for genetic |
| | | | | confinement systems |
| WHOLE | Germination | Slow | a portion of the seed | Useful for making lethal |
| SEEDLING | | Germination | germinates | plants for genetic |
| | | | significantly later | confinement systems |
| | | | than the rest of the | |
| | | | seed in the pot | |
| ROSETTE | Vitrified | Vitrified | leaves are somewhat | Useful for making lethal |
| LEAVES | | | translucent or ?water | plants for genetic |
| | | | soaked? | confinement systems |
| CAULINE | Vitrified | Vitrified | leaves are somewhat | Useful for making lethal |
| LEAVES | | | translucent or ?water | plants for genetic |
| | | | soaked? | confinement systems |
| COTYLEDONS | Albino | Opaque Albino | plant is opaque and | Useful for making lethal |
| | | | devoid of pigment | plants for genetic |
| | | | | confinement systems |
| COTYLEDONS | Albino | Translucent | plant is translucent | Useful for making lethal |
| | | Albino | and devoid of | plants for genetic |
| | | | pigment | confinement systems |
| WHOLE | Lethal | Seedling Lethal | cotyledons emerge | Useful for making lethal |
| SEEDLING | | | (although they are | plants for genetic |
| | | | often small), but then | confinement systems |
| | | | the plant ceases to | |
| | | | develop further; No | |
| | | | true leaves appear | |
| | | | and the plant dies | |
| | | | early (These differ | |
| | | | from yellow-green | |
| | | | lethals in that the | |
| | | | cotyledons are wild- | |
| | | | type in color and may | |
| | | | not look differ | |
| WHOLE | Lethal | Yellow-Green | cotyledons are small | Useful for making lethal |
| SEEDLING | | Lethal | and pale yellow- | plants for genetic |
| | | | green in color, but | confinement systems |
| | | | NOT totally devoid | |
| | | | of pigment; In | |
| | | | addition to yellow- | |
| | | | green cotyledons, | |
| | | | these plants produce | |
| | | | no or severely | |
| | | | reduced size true | |
| | | | leaves, which, if | |
| | | | present, are also | |
| | | | yellow-green; These | |
| | | | plants die prem | |
| WHOLE | Meristem Mutant | Meristem Mutant | this term | Useful for making lethal |
| SEEDLING | | | encompasses a | plants for genetic |
| | | | variety of | confinement systems |
| | | | phenotypes, all of | |
| | | | which have one thing | |
| | | | in common, i.e., they | |
| | | | all have something | |
| | | | significantly wrong | |
| | | | with how the | |
| | | | meristem is | |
| | | | producing its leaves; | |
| | | | Depending on the | |
| | | | severity of the | |
| | | | phenotype, the plants | |
| | | | in this category | |
| WHOLE | Seedling | Seedling | this term | Useful for making lethal |
| SEEDLING | Defective | Defective | encompasses a | plants for genetic |
| | | | variety of phenotypes | confinement systems |
| | | | which share similar | |
| | | | characteristics, i.e., | |
| | | | they are small, have | |
| | | | distorted structures, | |
| | | | and are prone to early | |
| | | | death; For example, | |
| | | | patterning mutants | |
| | | | would be a class of | |
| | | | mutants which fall | |
| | | | under this category | |
| WHOLE | Color | Yellow-Green | the leaves and | Useful for making lethal |
| PLANT | | Viable 1 | cotyledons are | plants for genetic |
| | | | yellow-green in | confinement systems |
| | | | color, but this is not a | |
| | | | lethal phenotype | |
| WHOLE | Color | Yellow-Green | the leaves are yellow- | Useful for making lethal |
| PLANT | | Viable 2 | green in color but the | plants for genetic |
| | | | cotyledons are a | confinement systems |
| | | | wild-type green in | |
| | | | color | |
| WHOLE | Color | Yellow-Green | the leaves start out | Useful for making lethal |
| PLANT | | Viable 3 | wild-type green and | plants for genetic |
| | | | gradually turn | confinement systems |
| | | | yellow-green in | |
| | | | color, while the | |
| | | | cotyledons stay wild- | |
| | | | type green | |
| WHOLE | Color | Yellow-Green | the leaves appear | Useful for making lethal |
| PLANT | | Viable 4 | wild-type green, but | plants for genetic |
| | | | slowly turn yellow- | confinement systems |
| | | | green over time, | |
| | | | while the cotyledons | |
| | | | appear and remain | |
| | | | yellow-green | |
| WHOLE | Stress | Seed Bleaching | Identify plants whose | Useful for making low |
| PLANT | | | seed coats do not | fiber seeds with increased |
| | | | bleach out under long | digestability |
| | | | bleach soaking | |
| ROSETTE | Fused | Leaf Fused to | the leaf is fused to an | Useful for making |
| LEAVES | | Inflorescence | inflorescence | ornamental plants with |
| | | | | flowers and leaves fused |
| ROSETTE | Interveinal | Interveinal | the leaf tissue is | Useful for making |
| LEAVES | Chlorosis | Chlorosis | chlorotic between its | ornamental plants with |
| | | | veins | modified color |
| CAULINE | Interveinal | Interveinal | the leaf tissue is | Useful for making |
| LEAVES | Chlorosis | Chlorosis | chlorotic between its | ornamental plants with |
| | | | veins | modified color |
| FLOWER | Organ | Fused Sepals | the sepals are fused | Useful for making |
| | Morphology | | together and won?t | ornamental plants with |
| | | | open naturally, but | modified flowers |
| | | | the flower is | |
| | | | otherwise wild-type | |
| FLOWER | Organ | Narrow Petals | the petals are | Useful for making |
| | Morphology | | abnormally narrow | ornamental plants with |
| | | | | modified flowers |
| FLOWER | Organ | Narrow Sepals | the sepals are | Useful for making |
| | Morphology | | abnormally narrow | ornamental plants with |
| | | | | modified flowers |
| FLOWER | Organ | Short Petals | the petals are | Useful for making |
| | Morphology | | abnormally short | ornamental plants with |
| | | | | modified flowers |
| FLOWER | Organ | Short Sepals | the sepals are | Useful for making |
| | Morphology | | abnormally short | ornamental plants with |
| | | | | modified flowers |
| FLOWER | Size | Large | flower is abnormally | Useful for making |
| | | | large (the percent | ornamental plants with |
| | | | difference in size | modified flowers |
| | | | compared to the | |
| | | | control should be | |
| | | | noted in the | |
| | | | comments) | |
| FLOWER | Size | Small | flower is abnormally | Useful for making |
| | | | small (the percent | ornamental plants with |
| | | | difference in size | modified flowers |
| | | | compared to the | |
| | | | control should be | |
| | | | noted in the | |
| | | | comments) | |
| FLOWER | Other | Other | this correlates with | Useful for making |
| | | | any flower mutant | ornamental plants with |
| | | | phenotypes which do | modified flowers |
| | | | not fit into the above | |
| | | | categories (a picture | |
| | | | should be taken for | |
| | | | documentation) | |
| INFLORESCENCE | Aerial Rosette | Aerial Fosette | rosette forms at or | Useful for making |
| | | | above the first | ornamental plants with |
| | | | internode | modified flowers |
| INFLORESCENCE | Appearance | Corkscrew | the inflorescence is | Useful for making |
| | | Appearance | really twisted, almost | ornamental plants with |
| | | | like a corkscrew, but | modified flowers |
| | | | somewhat more | |
| | | | irregular | |
| INFLORESCENCE | Appearance | Curved | the inflorescence has | Useful for making |
| | | Appearance | a slight, irregular | ornamental plants with |
| | | | curve upwards, | modified flowers |
| | | | greater than that of | |
| | | | the control plants | |
| INFLORESCENCE | Appearance | Multi- | the inflorescence is | Useful for making |
| | | Inflorescence | fused to another | ornamental plants with |
| | | Fusion | inflorescence, | modified flowers |
| | | | creating a celery-like | |
| | | | appearance | |
| INFLORESCENCE | Appearance | Undulate | the inflorescence is | Useful for making |
| | | Appearance | wavy in appearance | ornamental plants with |
| | | | | modified flowers |
| INFLORESCENCE | Branching | Acauline | first branching is not | Useful for making |
| | | Branching | subtended by a | ornamental plants with |
| | | | cauline leaf | modified flowers |
| INFLORESCENCE | Wax | Glaucous | inflorescence is | Useful for making |
| | | | abnormally dull in | ornamental plants with |
| | | | appearance | modified flowers |
| INFLORESCENCE | Wax | Glossy | inflorescence is | Useful for making |
| | | | shiny/glossy in | ornamental plants with |
| | | | appearance | modified flowers |
| INFLORESCENCE | Other | Other | this correlates with | Useful for making |
| | | | any inflorescence | ornamental plants with |
| | | | mutant phenotypes | modified flowers |
| | | | which do not fit into | |
| | | | the above categories | |
| | | | (a picture should be | |
| | | | taken for | |
| | | | documentation) | |
| COTYLEDONS | Asymmetric | Asymmetric | the shape of the | Useful for making |
| | | | cotyledon is | ornamental plants with |
| | | | asymmetric in | modified foliage |
| | | | reference to the | |
| | | | vertical axis | |
| ROSETTE | Other | Other | this correlates with | Useful for making |
| LEAVES | | | any leaf mutant | ornamental plants with |
| | | | phenotypes which do | modified leaves |
| | | | not fit into the above | |
| | | | categories (a picture | |
| | | | should be taken for | |
| | | | documentation) | |
| CAULINE | Other | Other | this correlates with | Useful for making |
| LEAVES | | | any cauline mutant | ornamental plants with |
| | | | phenotypes which do | modified leaves |
| | | | not fit into the above | |
| | | | categories (a picture | |
| | | | should be taken for | |
| | | | documentation) | |
| FLOWER | Homeotic | Homeotic | the flower has one or | Useful for making plants |
| | Mutant | Mutant | more of its organs | sterile and for genetic |
| | | | converted to another | confinement |
| | | | type of organ | |
| | | | (specific details | |
| | | | should be noted in | |
| | | | the comments) | |
| FLOWER | Organ | Aberrant Organ | there is an abnormal | Useful for making plants |
| | Morphology | Number | number of some or | sterile and for genetic |
| | | | all of the flowers | confinement |
| | | | organs | |
| FLOWER | Organ | Short Stamens | the stamens are | Useful for making plants |
| | Morphology | | abnormally short; | sterile and for genetic |
| | | | This often leads to | confinement |
| | | | mechanical problems | |
| | | | with fertility | |
| FLOWER | Fertility | Aborted fertility | the ovule is | Useful for making plants |
| | | | unfertilized and | sterile and for genetic |
| | | | appears as a brown or | confinement |
| | | | white speck in the | |
| | | | mature silique | |
| FLOWER | Fertility | Female-sterile | there is a problem | Useful for making plants |
| | | | with the ovules such | sterile and for genetic |
| | | | that no fertilization is | confinement |
| | | | occurring | |
| FLOWER | Fertility | Male-sterile | there is a problem | Useful for making plants |
| | | | with the pollen such | sterile and for genetic |
| | | | that no fertilization is | confinement |
| | | | occurring | |
| FLOWER | Fertility | Reduced fertility | a reduced number of | Useful for making plants |
| | | | successful | sterile and for genetic |
| | | | fertilization events, | confinement |
| | | | and therefore seeds, | |
| | | | are being produced | |
| | | | by the plant | |
| FLOWER | Fertility | Sterile | no successful | Useful for making plants |
| | | | fertilization events, | sterile and for genetic |
| | | | and therefore no seed | confinement |
| | | | is being produced by | |
| | | | the plant; The reason | |
| | | | for this sterility is not | |
| | | | known at the time of | |
| | | | the observation | |
| FLOWER | Fertility | Other | this correlates with | Useful for making plants |
| | | | any fertility mutant | sterile and for genetic |
| | | | phenotypes which do | confinement |
| | | | not fit into the above | |
| | | | categories (a picture | |
| | | | should be taken for | |
| | | | documentation) | |
| WHOLE | Stress | Early Flowering | Identify plants that | Useful for making plants |
| PLANT | | | flower early | that flower early |
| COTYLEDONS | Petiole Length | Long Petioles | the cotyledon petioles | Useful for making plants |
| | | | are abnormally long | that grow and better in |
| | | | (the percent | shade |
| | | | difference in size | |
| | | | compared to the | |
| | | | control should be | |
| | | | noted in the | |
| | | | comments) | |
| ROSETTE | Petiole Length | Varying Petiole | the leaf petioles vary | Useful for making plants |
| LEAVES | | Lengths | in length throughout | that grow better in shade |
| | | | the rosette | |
| ROSETTE | Petiole Length | Long Petioles | the leaf petioles are | Useful for making plants |
| LEAVES | | | abnormally long (the | that grow better in shade |
| | | | percent difference in | |
| | | | size compared to the | |
| | | | control should be | |
| | | | noted in the | |
| | | | comments) | |
| | | | | Useful for making plants |
| | | | | tolerant to biotic stress |
| WHOLE | Stress | | Identify plants able to | Useful for making plants |
| PLANT | | | tolerate high density | tolerant to density and |
| | | | and no phosphate and | low fertilizer |
| | | | nitrogen, possible | |
| | | | lead assay for vigor | |
| | | | under population | |
| | | | density and low | |
| | | | nutrient conditions | |
| WHOLE | Stress | pH (high) | Identify plants | Useful for making plants |
| PLANT | | | tolerant to high pH, | tolerant to high pH or low |
| | | | and possibly low | phosphate |
| | | | phosphate | |
| WHOLE | Stress | Low Nitrate | Identify plants | Useful for making plants |
| PLANT | | | tolerant to low | tolerant to low nitrogen |
| | | | nitrogen/nitrate | |
| | | | growth media | |
| WHOLE | Stress | LNABA | Identify plants | Useful for making plants |
| PLANT | | | tolerant to low | tolerant to low nitrogen |
| | | | nitrogen and high | |
| | | | ABA concentrations | |
| WHOLE | Stress | No Nitrogen | Identify plants with | Useful for making plants |
| PLANT | | | increased vigor under | tolerant to low nitrogen |
| | | | no nitrogen | |
| | | | conditions | |
| WHOLE | Stress | MSX | Identify plants | Useful for making plants |
| PLANT | | | tolerant to nitrogen | tolerant to low nitrogen |
| | | | assimilation inhibitor, | |
| | | | and possibly low | |
| | | | nitrogen tolerance | |
| | | | and/or seed nitrogen | |
| | | | accumulation | |
| WHOLE | Stress | No N, No PO4 | Identify plants | Useful for making plants |
| PLANT | | | tolerant to no | tolerant to low |
| | | | nitrogen and no | nitrogen/low phosphate |
| | | | phosphate growth | |
| | | | media | |
| WHOLE | Stress | Oxidative | Identify plants | Useful for making plants |
| PLANT | | | tolerant to oxidative | tolerant to oxidative |
| | | | stress | stresses |
| ROSETTE | Trichomes | Few Trichomes | trichomes are sparse | Useful for making plants |
| LEAVES | | | but present on the | with enhanced chemical |
| | | | leaves | composition |
| ROSETTE | Trichomes | Glabrous | trichomes are totally | Useful for making plants |
| LEAVES | | | absent | with enhanced chemical |
| | | | | composition |
| ROSETTE | Trichomes | Abnormal | the trichomes are | Useful for making plants |
| LEAVES | | Trichome Shape | abnormally shaped | with enhanced chemical |
| | | | | composition |
| CAULINE | Trichomes | Few Trichomes | trichomes are sparse | Useful for making plants |
| LEAVES | | | but present on the | with enhanced chemical |
| | | | leaves | composition |
| CAULINE | Trichomes | Glabrous | trichomes are totally | Useful for making plants |
| LEAVES | | | absent | with enhanced chemical |
| | | | | composition |
| CAULINE | Trichomes | Abnormal | the trichomes are | Useful for making plants |
| LEAVES | | Trichome Shape | abnormally shaped | with enhanced chemical |
| | | | | composition |
| INFLORESCENCE | Trichomes | Glabrous | trichomes are totally | Useful for making plants |
| | | | absent | with enhanced chemical |
| | | | | composition |
| INFLORESCENCE | Trichomes | Abnormal | the trichomes are | Useful for making plants |
| | | Trichome Shape | abnormally shaped | with enhanced chemical |
| | | | | composition |
| ROSETTE | Curled | Corkscrew | leaves appear as | Useful for making plants |
| LEAVES | | | “Curled 5”, with the | with altered leaf shape eg |
| | | | additional attribute of | curled leaves |
| | | | twisting like a | |
| | | | corkscrew, instead of | |
| | | | uniformly curling | |
| | | | from both sides of the | |
| | | | leaf | |
| ROSETTE | Curled | Cup-shaped | leaves are curled up | Useful for making plants |
| LEAVES | | | at the leaf margins | with altered leaf shape eg |
| | | | such that they form a | curled leaves |
| | | | cup or bowl-like | |
| | | | shape | |
| ROSETTE | Curled | Curled 1 | leaves are abnormally | Useful for making plants |
| LEAVES | | | curled slightly up or | with altered leaf shape eg |
| | | | down at the leaf | curled leaves |
| | | | margins, but do not | |
| | | | fall under the “cup- | |
| | | | shaped” description | |
| | | | (least severe type) | |
| ROSETTE | Curled | Curled 2 | leaves are abnormally | Useful for making plants |
| LEAVES | | | curled up or down at | with altered leaf shape eg |
| | | | the leaf margins, but | curled leaves |
| | | | do not fall under the | |
| | | | “cup-shaped” | |
| | | | description (more | |
| | | | severe than Curled 1, | |
| | | | but less severe than | |
| | | | Curled 3) | |
| ROSETTE | Curled | Curled 3 | leaves are abnormally | Useful for making plants |
| LEAVES | | | curled up or down at | with altered leaf shape eg |
| | | | the leaf margins, but | curled leaves |
| | | | do not fall under the | |
| | | | “cup-shaped” | |
| | | | description (more | |
| | | | severe than Curled 2, | |
| | | | but less severe than | |
| | | | Curled 4) | |
| ROSETTE | Curled | Curled 4 | leaves are abnormally | Useful for making plants |
| LEAVES | | | curled/rolled up or | with altered leaf shape eg |
| | | | down at the leaf | curled leaves |
| | | | margins (more | |
| | | | severe than Curled 3, | |
| | | | but less severe than | |
| | | | Curled 5) | |
| ROSETTE | Curled | Curled 5 | leaves are completely | Useful for making plants |
| LEAVES | | | curled/rolled up or | with altered leaf shape eg |
| | | | down at the leaf | curled leaves |
| | | | margins (most severe | |
| | | | type) | |
| CAULINE | Curled | Corkscrew | leaves appear as | Useful for making plants |
| LEAVES | | | “Curled 5”, with the | with altered leaf shape eg |
| | | | additional attribute of | curled leaves |
| | | | twisting like a | |
| | | | corkscrew, instead of | |
| | | | uniformly curling | |
| | | | from both sides of the | |
| | | | leaf | |
| CAULINE | Curled | Cup-shaped | the cauline leaves are | Useful for making plants |
| LEAVES | | | curled up at the leaf | with altered leaf shape eg |
| | | | margins such that | curled leaves |
| | | | they form a cup or | |
| | | | bowl-like shape | |
| CAULINE | Curled | Curled 1 | the cauline leaves are | Useful for making plants |
| LEAVES | | | abnormally curled | with altered leaf shape eg |
| | | | slightly up or down at | curled leaves |
| | | | the leaf margins, but | |
| | | | do not fall under the | |
| | | | “cup-shaped” | |
| | | | description (least | |
| | | | severe type) | |
| CAULINE | Curled | Curled 2 | the cauline leaves are | Useful for making plants |
| LEAVES | | | abnormally curled up | with altered leaf shape eg |
| | | | or down at the leaf | curled leaves |
| | | | margins, but do not | |
| | | | fall under the “cup- | |
| | | | shaped” description | |
| | | | (more severe than | |
| | | | Curled 1, but less | |
| | | | severe than Curled 3) | |
| CAULINE | Curled | Curled 3 | the cauline leaves are | Useful for making plants |
| LEAVES | | | abnormally curled up | with altered leaf shape eg |
| | | | or down at the leaf | curled leaves |
| | | | margins, but do not | |
| | | | fall under the “cup- | |
| | | | shaped” description | |
| | | | (more severe than | |
| | | | Curled 2, but less | |
| | | | severe than Curled 4) | |
| CAULINE | Curled | Curled 4 | the cauline leaves are | Useful for making plants |
| LEAVES | | | abnormally | with altered leaf shape eg |
| | | | curled/rolled up or | curled leaves |
| | | | down at the leaf | |
| | | | margins (more | |
| | | | severe than Curled 3, | |
| | | | but less severe than | |
| | | | Curled 5) | |
| CAULINE | Curled | Curled 5 | the cauline leaves are | Useful for making plants |
| LEAVES | | | completely | with altered leaf shape eg |
| | | | curled/rolled up or | curled leaves |
| | | | down at the leaf | |
| | | | margins (most severe | |
| | | | type) | |
| ROSETTE | Size | Small | rosette leaves are | Useful for making plants |
| LEAVES | | | abnormally small | with decreased vegetative |
| | | | (the percent | growth |
| | | | difference in size | |
| | | | compared to the | |
| | | | control should be | |
| | | | noted in the | |
| | | | comments) | |
| COTYLEDONS | Wilted | Wilted | cotyledons appear | Useful for making plants |
| | | | wilted, i.e., they look | with enhanced abiotic |
| | | | as though they have | stress tolerance |
| | | | suffered from | |
| | | | drought conditions | |
| ROSETTE | Wax | Glaucous | leaves are abnormally | Useful for making plants |
| LEAVES | | | dull in appearance | with enhanced abiotic |
| | | | | stress tolerance |
| ROSETTE | Wax | Glossy | leaves are | Useful for making plants |
| LEAVES | | | shiny/glossy in | with enhanced abiotic |
| | | | appearance | stress tolerance |
| CAULINE | Wax | Glaucous | leaves are abnormally | Useful for making plants |
| LEAVES | | | dull in appearance | with enhanced abiotic |
| | | | | stress tolerance |
| CAULINE | Wax | Glossy | leaves are | Useful for making plants |
| LEAVES | | | shiny/glossy in | with enhanced abiotic |
| | | | appearance | stress tolerance |
| WHOLE | Stress | Metabolic | Identify plants with | Useful for making plants |
| PLANT | | Profiling | altered metabolic | with enhanced metabolite |
| | | | profiles as defined in | accumulation |
| | | | 4a | |
| WHOLE | Stress | Plant | Identify plants with | Useful for making plants |
| PLANT | | Architecture | improved architecture | with enhanced plant |
| | | | | architecture |
| WHOLE | Stress | ABA | Identify plants | Useful for making plants |
| PLANT | | | tolerant to ABA, and | with enhanced tolerance |
| | | | possibly drought | to drought |
| | | | and/or other stresses | |
| WHOLE | Stress | Mannitol | Identify plants | Useful for making plants |
| PLANT | | | tolerant to mannitol, | with enhanced tolerance |
| | | | and possibly drought | to drought |
| | | | stress | |
| WHOLE | Stress | Dessication | Identify plants | Useful for making plants |
| PLANT | | | tolerant to water loss, | with enhanced tolerance |
| | | | possibly drought | to drought |
| | | | stress tolerant | |
| WHOLE | Stress | High Sucrose | Identify plants | Useful for making plants |
| PLANT | | | tolerant to high | with enhanced tolerance |
| | | | sucrose conditions | to drought |
| | | | (possible Lead assay | |
| | | | for C/N partitioning) | |
| WHOLE | Stress | Heat | Identify plants with | Useful for making plants |
| PLANT | | | thermotolerance | with enhanced tolerance |
| | | | | to heat |
| WHOLE | Stress | High Nitrogen | Identify plants | Useful for making plants |
| PLANT | | | tolerant to high | with enhanced tolerance |
| | | | nitrogen conditions | to high nitrogen |
| WHOLE | Stress | Etiolation | Identify plants with | Useful for making plants |
| PLANT | | | increased vigor in the | with enhanced tolerance |
| | | | dark | to light stress |
| ROSETTE | Disorganized | Disorganized | rosette leaves do not | Useful for making plants |
| LEAVES | Rosette | Rosette | appear in the normal | with increased biomass |
| | | | fashion, i.e., their | |
| | | | phyllotaxy may be | |
| | | | abnormal or too | |
| | | | many leaves may be | |
| | | | emerging in | |
| | | | comparison to the | |
| | | | control | |
| INFLORESCENCE | Phyllotaxy | Even Phyllotaxy | a phyllotaxy mutant | Useful for making plants |
| | | | whose new branches | with increased biomass |
| | | | emerge at exactly the | |
| | | | same height as each | |
| | | | other, i.e., there is no | |
| | | | internode between | |
| | | | them | |
| COTYLEDONS | Shape | Elliptic Shape | cotyledons are quite | Useful for making plants |
| | | | narrow and pointed, | with increased biomass |
| | | | more so than | and foliage |
| | | | lanceolate | |
| ROSETTE | Fused | Leaf Fused to | the leaf is fused to its | Useful for making plants |
| LEAVES | | Petiole | petiole | with increased biomass |
| | | | | and foliage |
| ROSETTE | Shape | Cordate Shaped | similar to ovate, | Useful for making plants |
| LEAVES | | | except the leaf is not | with increased biomass |
| | | | rounded at its base | and foliage |
| ROSETTE | Shape | Elliptic Shaped | leaves are quite | Useful for making plants |
| LEAVES | | | narrow and pointed, | with increased biomass |
| | | | more so that | and foliage |
| | | | lanceolate | |
| ROSETTE | Shape | Lanceolate | leaves are narrow and | Useful for making plants |
| LEAVES | | Shaped | come to a dull point | with increased biomass |
| | | | at the apex | and foliage |
| ROSETTE | Shape | Lobed Shaped | leaves have very deep | Useful for making plants |
| LEAVES | | | and rounded | with increased biomass |
| | | | serrations, giving an | and foliage |
| | | | appearance of many | |
| | | | lobes forming the | |
| | | | margins of the leaves | |
| ROSETTE | Shape | Oval Shaped | leaves are much | Useful for making plants |
| LEAVES | | | rounder than wild- | with increased biomass |
| | | | type | and foliage |
| ROSETTE | Shape | Ovate Shaped | leaves are wider at | Useful for making plants |
| LEAVES | | | base than at apex, | with increased biomass |
| | | | otherwise similar to | and foliage |
| | | | wild-type | |
| ROSETTE | Shape | Serrate Margins | leaf margins have | Useful for making plants |
| LEAVES | | | little ?teeth? on them, | with increased biomass |
| | | | i.e., they are serrated | and foliage |
| ROSETTE | Shape | Trident Shaped | leaves look | Useful for making plants |
| LEAVES | | | somewhat like a | with increased biomass |
| | | | trident, i.e., they have | and foliage |
| | | | a sharp point at the | |
| | | | apex, and a sharp | |
| | | | point on each side | |
| ROSETTE | Shape | Undulate Shaped | leaves are wavy | Useful for making plants |
| LEAVES | | | | with increased biomass |
| | | | | and foliage |
| WHOLE | Rosette Shape | Bushy Rosette | the different petioles | Useful for making plants |
| PLANT | | Shaped | have very varied | with increased biomass |
| | | | liminal angles, giving | and foliage |
| | | | the plant a very | |
| | | | bushy appearance; | |
| | | | This is often | |
| | | | accompanied by a | |
| | | | “Disorganized | |
| | | | Rosette” phenotype | |
| WHOLE | Rosette Shape | Flat Rosette | the petioles have a | Useful for making plants |
| PLANT | | Shaped | very small liminal | with increased biomass |
| | | | angle, i.e., the rosette | and foliage |
| | | | appears flat instead of | |
| | | | having its usual slight | |
| | | | vertical angle | |
| WHOLE | Rosette Shape | Standing Rosette | the petioles have a | Useful for making plants |
| PLANT | | Shaped | very large liminal | with increased biomass |
| | | | angle, i.e., it appears | and foliage |
| | | | as though the leaves | |
| | | | are standing up | |
| | | | instead of having | |
| | | | their usual small | |
| | | | vertical angle from | |
| | | | the soil | |
| CAULINE | Fused | Leaf Fused to | the cauline leaf is | Useful for making plants |
| LEAVES | | Inflorescence | fused to an | with increased biomass |
| | | | inflorescence or | and foliage |
| | | | branch | |
| CAULINE | Fused | Leaf Fused to | the cauline leaf is | Useful for making plants |
| LEAVES | | Leaf | fused to itself or | with increased biomass |
| | | | another cauline leaf | and foliage |
| CAULINE | Shape | Cordate Shaped | similar to ovate, | Useful for making plants |
| LEAVES | | | except the leaf is not | with increased biomass |
| | | | rounded at its base | and foliage |
| CAULINE | Shape | Elliptic Shaped | leaves are quite | Useful for making plants |
| LEAVES | | | narrow and pointed, | with increased biomass |
| | | | more so that | and foliage |
| | | | lanceolate | |
| CAULINE | Shape | Lanceolate | leaves are narrow and | Useful for making plants |
| LEAVES | | Shaped | come to a dull point | with increased biomass |
| | | | at the apex | and foliage |
| CAULINE | Shape | Lobed Shaped | leaves have very deep | Useful for making plants |
| LEAVES | | | and rounded | with increased biomass |
| | | | serrations, giving an | and foliage |
| | | | appearance of many | |
| | | | lobes forming the | |
| | | | margins of the leaves | |
| CAULINE | Shape | Oval Shaped | leaves are much | Useful for making plants |
| LEAVES | | | rounder than wild- | with increased biomass |
| | | | type | and foliage |
| CAULINE | Shape | Ovate Shaped | leaves are wider at | Useful for making plants |
| LEAVES | | | base than at apex, | with increased biomass |
| | | | otherwise similar to | and foliage |
| | | | wild-type | |
| CAULINE | Shape | Serrate Margins | leaf margins have | Useful for making plants |
| LEAVES | | | little ?teeth? on them, | with increased biomass |
| | | | i.e., they are serrated | and foliage |
| CAULINE | Shape | Trident Shaped | leaves look | Useful for making plants |
| LEAVES | | | somewhat like a | with increased biomass |
| | | | trident, i.e., they have | and foliage |
| | | | a sharp point at the | |
| | | | apex, and a sharp | |
| | | | point on each side | |
| CAULINE | Shape | Undulate Shaped | leaves are wavy | Useful for making plants |
| LEAVES | | | | with increased biomass |
| | | | | and foliage |
| CAULINE | Size | Large | cauline is abnormally | Useful for making plants |
| LEAVES | | | large (the percent | with increased biomass |
| | | | difference in size | and foliage |
| | | | compared to the | |
| | | | control should be | |
| | | | noted in the | |
| | | | comments) | |
| CAULINE | Size | Small | cauline is abnormally | Useful for making plants |
| LEAVES | | | small (the percent | with increased biomass |
| | | | difference in size | and foliage |
| | | | compared to the | |
| | | | control should be | |
| | | | noted in the | |
| | | | comments) | |
| LATERAL | Length | Smaller Lateral | the lateral roots are | Useful for making plants |
| ROOTS | | Root | abnormally short | with increased root |
| | | | | growth to prevent lodging |
| | | | | or enhance nutrient |
| | | | | uptake |
| PRIMARY | Length | Long Primary | the primary root is | Useful for making plants |
| ROOT | | Root | abnormally long | with increased root |
| | | | (the percent | growth to prevent lodging |
| | | | difference in size | or enhance nutrient |
| | | | compared to the | uptake |
| | | | control should be | |
| | | | noted in the | |
| | | | comments) | |
| PRIMARY | Length | Short Primary | the primary root is | Useful for making plants |
| ROOT | | Root | abnormally short | with increased root |
| | | | (the percent | growth to prevent lodging |
| | | | difference in size | or enhance nutrient |
| | | | compared to the | uptake |
| | | | control should be | |
| | | | noted in the | |
| | | | comments) | |
| WHOLE | Stress | Plant Size | Identify plants of | Useful for making plants |
| PLANT | | | increased size | with increased size and |
| | | | compared to wild | biomass |
| | | | type | |
| WHOLE | Stress | Starch | Identify plants with | Useful for making plants |
| PLANT | | | increased starch | with increased starch |
| | | | accumulation | content |
| WHOLE | Stress | Cold | Identify plants that | Useful for making plants |
| PLANT | | Germination | germinate better at | with increased tolerance |
| | | | cold temperatures | to cold stress |
| WHOLE | Stress | Cold Growth | Identify plants that | Useful for making plants |
| PLANT | | | grow faster at cold | with increased tolerance |
| | | | temperatures | to cold stress |
| WHOLE | Stress | Soil Drought | Identify plants with | Useful for making plants |
| PLANT | | | increased tolerance to | with increased tolerance |
| | | | soil drought | to drought |
| WHOLE | Stress | Soil Drought - | Identify plants that | Useful for making plants |
| PLANT | | Desiccation | are tolerant to low | with increased tolerance |
| | | tolerance | soil moisture and | to drought |
| | | | resist wilting | |
| WHOLE | Stress | PEG | Identify plants | Useful for making plants |
| PLANT | | | tolerant to PEG, and | with increased tolerance |
| | | | possibly drought | to drought |
| | | | stress | |
| SEED | Size | Large | the seed is | Useful for making plants |
| | | | abnormally large | with larger seeds |
| | | | (the percent | |
| | | | difference in size | |
| | | | compared to the | |
| | | | control should be | |
| | | | noted in the | |
| | | | comments) | |
| INFLORESCENCE | Branching | Asecondary | the plant does not | Useful for making plants |
| | | Branching | form any secondary | with modified flowers |
| | | | inflorescences | |
| SEED | Size | Small | the seed is | Useful for making plants |
| | | | abnormally small | with smaller seeds or no |
| | | | (the percent | seeds |
| | | | difference in size | |
| | | | compared to the | |
| | | | control should be | |
| | | | noted in the | |
| | | | comments) | |
| WHOLE | Stress | C/N Content | Identify plants/seeds | Useful for making seeds |
| PLANT | | | with altered | with altered |
| | | | carbon/nitrogen | carbon/nitrogen levels |
| | | | levels | |
| INFLORESCENCE | Internode Length | Short Internode | the internode is | Useful for making shorter |
| | | | abnormally short | plants and plants with |
| | | | (the percent | modified flowers |
| | | | difference in length | |
| | | | compared to the | |
| | | | control should be | |
| | | | noted in the | |
| | | | comments) | |
| WHOLE | Dwarf | Brassino-Steroid | these plants are small | Useful for making smaller |
| PLANT | | Dwarf | in stature, dark green, | plants |
| | | | have oval leaves, | |
| | | | strong bolts, and are | |
| | | | often sterile | |
| WHOLE | Dwarf | Misc. Dwarf | these are dwarf plants | Useful for making smaller |
| PLANT | | | the do not fall under | plants |
| | | | the brassino-steroid | |
| | | | dwarf category | |
| HYPOCOTYL | Length | Short | hypocotyl is visibly | Useful for making smaller |
| | | | shorter than in wild- | plants |
| | | | type (the percent | |
| | | | difference in size | |
| | | | compared to the | |
| | | | control should be | |
| | | | noted in the | |
| | | | comments) | |
| INFLORESCENCE | Height | Short | the inflorescences of | Useful for making smaller |
| | | | the plants are | plants |
| | | | abnormally short | |
| | | | (plant height is | |
| | | | encompassed under | |
| | | | the whole plant size | |
| | | | category, but this | |
| | | | entry would be used | |
| | | | if the height of the | |
| | | | plant is abnormal, but | |
| | | | is otherwise of | |
| | | | normal size) (the | |
| | | | percent difference in | |
| | | | size | |
| WHOLE | Size | Small | plant is abnormally | Useful for making smaller |
| SEEDLING | | | small (the percent | plants |
| | | | difference in size | |
| | | | compared to the | |
| | | | control should be | |
| | | | noted in the | |
| | | | comments) | |
| ROSETTE | Petiole Length | Short Petioles | the leaf petioles are | Useful for making smaller |
| LEAVES | | | abnormally short | plants |
| | | | (the percent | |
| | | | difference in size | |
| | | | compared to the | |
| | | | control should be | |
| | | | noted in the | |
| | | | comments) | |
| WHOLE | Size | Small | plant is abnormally | Useful for making smaller |
| PLANT | | | small (the percent | plants |
| | | | difference in size | |
| | | | compared to the | |
| | | | control should be | |
| | | | noted in the | |
| | | | comments) | |
| CAULINE | Petiole Length | Short Petioles | the cauline petioles | Useful for making smaller |
| LEAVES | | | are abnormally short | plants |
| | | | (the percent | |
| | | | difference in size | |
| | | | compared to the | |
| | | | control should be | |
| | | | noted in the | |
| | | | comments) | |
| INFLORESCENCE | Strength | Strong | the primary | Useful for making |
| | | | inflorescence appears | stronger plants |
| | | | significantly stronger, | |
| | | | whether by thickness | |
| | | | or rigidity | |
| INFLORESCENCE | Strength | Weak | the primary | Useful for making |
| | | | inflorescence appears | stronger plants |
| | | | significantly weaker, | |
| | | | whether by thickness | |
| | | | or rigidity | |
| INFLORESCENCE | Inflorescence | Thickness | thickness of the | Useful for making |
| | | | primary inflorescence | stronger plants |
| HYPOCOTYL | Length | Long | hypocotyl is visibly | Useful for making taller |
| | | | longer than in wild- | plants |
| | | | type (the percent | |
| | | | difference in size | |
| | | | compared to the | |
| | | | control should be | |
| | | | noted in the | |
| | | | comments) | |
| INFLORESCENCE | Internode Length | Long Internode | the internode is | Useful for making taller |
| | | | abnormally long (the | plants and plants with |
| | | | percent difference in | longer flowers |
| | | | length compared to | |
| | | | the control should be | |
| | | | noted in the | |
| | | | comments) | |
| INFLORESCENCE | Height | Tall | the inflorescences of | Useful for making taller |
| | | | the plants are | plants and plants with |
| | | | abnormally long | longer inflorescences |
| | | | (plant height is | |
| | | | encompassed under | |
| | | | the whole plant size | |
| | | | category, but this | |
| | | | entry would be used | |
| | | | if the height of the | |
| | | | plant is abnormal, but | |
| | | | is otherwise of | |
| | | | normal size) (the | |
| | | | percent difference in | |
| | | | size | |
| SEED | Color | Dark Color | the seed is | Useful for modifying |
| | | | abnormally dark | fiber content in seed |
| SEED | Color | Light Color | the seed is | Useful for modifying |
| | | | abnormally light; | fiber content in seed |
| | | | Transparent Testa is | |
| | | | an example of this | |
| | | | phenotype | |
| SILIQUES | Shape | Bent | the silique has sharp | Useful for modifying fruit |
| | | | bend to it part of the | shape, composition and |
| | | | way down the length | seed yield |
| | | | of the silique; this | |
| | | | bend can be as much | |
| | | | as approaching 90 | |
| | | | degrees | |
| SILIQUES | Shape | Bulging | the seeds in the | Useful for modifying fruit |
| | | | silique appears | shape, composition and |
| | | | “shrink-wrapped”, | seed yield |
| | | | giving the silique a | |
| | | | bulging appearance | |
| SILIQUES | Shape | Clubbed | the silique is | Useful for modifying fruit |
| | | | somewhat bulbous at | shape, composition and |
| | | | its terminal end | seed yield |
| SILIQUES | Shape | Sickle | the silique is curved, | Useful for modifying fruit |
| | | | much like the blade | shape, composition and |
| | | | of a sickle | seed yield |
| INFLORESCENCE | Branching | No Branching | there is no branching | Useful for modifying |
| | | | at all | plant architecture, ie |
| | | | | amount of branching |
| INFLORESCENCE | Branching | Horizontal | new branches arise at | Useful for modifying |
| | | Branching | a 90 degree angle | plant architecture, ie |
| | | | from the bolt they are | branch angle |
| | | | emerging from | |
| COTYLEDONS | Horizontally | Horizontally | cotyledon is visibly | Useful for modifying |
| | Oblong | Oblong | wider than it is long, | plant architecture, ie leaf |
| | | | and it is also | structure |
| | | | symmetrical (or very | |
| | | | close to it) when cut | |
| | | | along its horizontal | |
| | | | axis | |
| INFLORESCENCE | Branching | Two Leaf | two cauline leaves | Useful for modifying |
| | | Branching | subtend branches | plant architecture, ie |
| | | | instead of one | reducing foliage |
| INFLORESCENCE | Branching | Reduced Apical | the dominance of the | Useful for modifying |
| | | Dominance | primary inflorescence | plant structure, ie |
| | | | is diminished, with | increased branching |
| | | | the secondaries | |
| | | | appearing as | |
| | | | dominant or nearly as | |
| | | | dominant | |
| SEED | Seed | Stacked | the seeds/embryos | Useful for modifying seed |
| | Arrangement | Arrangement | are stacked one on | content |
| | | | top of the other | |
| | | | within the silique, | |
| | | | instead of having the | |
| | | | usual side-by-side | |
| | | | distribution | |
| SEED | Other | Other | this correlates with | Useful for modifying seed |
| | | | any seed mutant | content |
| | | | phenotypes which do | |
| | | | not fit into the above | |
| | | | categories (a picture | |
| | | | should be taken for | |
| | | | documentation) | |
| SEED | Shape | Oval Shape | the seeds are much | Useful for modifying seed |
| | | | more rounded on the | structure and composition |
| | | | ends, giving the seed | |
| | | | a true oval | |
| | | | appearance | |
| SEED | Shape | Ridged Shape | the seeds have small | Useful for modifying seed |
| | | | ridges or bumps on | structure and composition |
| | | | them | |
| SEED | Shape | Tapered Shape | the ends of the seeds | Useful for modifying seed |
| | | | narrow down to a | structure and composition |
| | | | much sharper point | |
| | | | than usual | |
| COTYLEDONS | Cotyledon | Single Cotyledon | Only one cotyledon | Useful for modifying seed |
| | Number | | appears after | structure and content |
| | | | germination; This is | |
| | | | simply one cotyledon | |
| | | | that had formed | |
| | | | instead of two, and is | |
| | | | not related to the | |
| | | | fused phenotype; | |
| | | | With this exception, | |
| | | | the plant is often | |
| | | | otherwise wild-type | |
| | | | in appearance | |
| COTYLEDONS | Cotyledon | Tricot | three cotyledons | Useful for modifying seed |
| | Number | | emerge instead of | structure and content |
| | | | two; With this | |
| | | | exception, the plant is | |
| | | | often otherwise wild- | |
| | | | type in appearance | |
| COTYLEDONS | Curled | Cup-shaped | cotyledons are curled | Useful for modifying seed |
| | | | up at the cotyledon | structure and content |
| | | | margins such that | |
| | | | they form a cup or | |
| | | | bowl-like shape | |
| COTYLEDONS | Curled | Curled 1 | cotyledons are | Useful for modifying seed |
| | | | abnormally curled | structure and content |
| | | | slightly up or down at | |
| | | | the cotyledon | |
| | | | margins, but do not | |
| | | | fall under the “cup- | |
| | | | shaped” description | |
| | | | (least severe type) | |
| COTYLEDONS | Curled | Curled 2 | cotyledons are | Useful for modifying seed |
| | | | abnormally curled up | structure and content |
| | | | or down at the | |
| | | | cotyledon margins, | |
| | | | but do not fall under | |
| | | | the “cup-shaped” | |
| | | | description (more | |
| | | | severe than Curled 1, | |
| | | | but less severe than | |
| | | | Curled 3) | |
| COTYLEDONS | Curled | Curled 3 | cotyledons are | Useful for modifying seed |
| | | | abnormally curled up | structure and content |
| | | | or down at the | |
| | | | cotyledon margins, | |
| | | | but do not fall under | |
| | | | the “cup-shaped” | |
| | | | description (more | |
| | | | severe than Curled 2, | |
| | | | but less severe than | |
| | | | Curled 4) | |
| COTYLEDONS | Curled | Curled 4 | cotyledons are | Useful for modifying seed |
| | | | abnormally | structure and content |
| | | | curled/rolled up or | |
| | | | down at the | |
| | | | cotyledon margins | |
| | | | (more severe than | |
| | | | Curled 3, but less | |
| | | | severe than Curled 5) | |
| COTYLEDONS | Curled | Curled 5 | cotyledons are | Useful for modifying seed |
| | | | completely | structure and content |
| | | | curled/rolled up or | |
| | | | down at the | |
| | | | cotyledon margins | |
| | | | (most severe type) | |
| COTYLEDONS | Dimorphic | Dimorphic | one cotyledon is | Useful for modifying seed |
| | Cotyledons | Cotyledons | significantly larger | structure and content |
| | | | than the other | |
| COTYLEDONS | Fused | Fused 1 | cotyledons are fused | Useful for modifying seed |
| | | | to each other, | structure and content |
| | | | creating one | |
| | | | cotyledon structure | |
| | | | (least severe type) | |
| COTYLEDONS | Fused | Fused 2 | cotyledons are fused | Useful for modifying seed |
| | | | to each other, | structure and content |
| | | | creating one | |
| | | | cotyledon structure | |
| | | | (more severe than | |
| | | | Fused 1, but less | |
| | | | severe than Fused 3) | |
| COTYLEDONS | Fused | Fused 3 | cotyledons are fused | Useful for modifying seed |
| | | | to each other, | structure and content |
| | | | creating one | |
| | | | cotyledon structure | |
| | | | (more severe than | |
| | | | Fused 2, but less | |
| | | | severe than Fused 4) | |
| COTYLEDONS | Fused | Fused 4 | cotyledons are fused | Useful for modifying seed |
| | | | to each other, | structure and content |
| | | | creating one | |
| | | | cotyledon structure | |
| | | | (more severe than | |
| | | | Fused 3, but less | |
| | | | severe than Fused 5) | |
| COTYLEDONS | Fused | Fused 5 | cotyledons are fused | Useful for modifying seed |
| | | | to each other, | structure and content |
| | | | creating one | |
| | | | cotyledon structure | |
| | | | (most severe type) | |
| COTYLEDONS | Other | Other | this correlates with | Useful for modifying seed |
| | | | any cotyledon mutant | structure and content |
| | | | phenotypes which do | |
| | | | not fit into the above | |
| | | | categories (a picture | |
| | | | should be taken for | |
| | | | documentation) | |
| ROSETTE | Fused | Leaf Fused to | the leaf is fused to | Useful for plants with |
| LEAVES | | Leaf | itself or another leaf | fused leaves eg |
| | | | | ornamentals |
| COTYLEDONS | Petiole Length | Short Petioles | the cotyledon petioles | Useful for shade |
| | | | are abnormally short | avoidance and for making |
| | | | (the percent | smaller plants |
| | | | difference in size | |
| | | | compared to the | |
| | | | control should be | |
| | | | noted in the | |
| | | | comments) | |
| PRIMARY | Agravitropic | Agravitropic | the primary root does | |
| ROOT | | | not appear to have a | |
| | | | gravitropic response | |
| PRIMARY | Kinked | Kinked | there is a sharp bend | |
| ROOT | | | in the root | |
| ROSETTE | Rosette Diameter | Diameter | diameter of rosette | |
| LEAVES | | | | |
| WHOLE | Plant Weight | Plant Weight | weight of whole plant | |
| PLANT | | | | |
| WHOLE | Plant Height | Height | height of whole plant | |
| PLANT | | | | |
| WHOLE | Plant DTH | Dth | days to harvest of | |
| PLANT | | | plant | |
| WHOLE | Plant Harvest | Harvest Index | harvest index of plant | |
| PLANT | Index | | | |
| CAULINE | Fused | Leaf Fused to | the cauline leaf is | |
| LEAVES | | Petiole | fused to its petiole | |
| N/A | N/A | N/A | N/A | |
| WHOLE | HERBICIDE | HERBICIDE | herbicide segregation | |
| PLANT | SEGREGATION | SEGREGATION | ratio | |
| WHOLE | N/A | No Mutant | The plants were | |
| PLANT | | Phenotype | screened at all | |
| | | Observed | appropriate stages | |
| | | | and showed no | |
| | | | mutant phenotype, | |
| | | | i.e., they looked like | |
| | | | normal, wild type | |
| | | | Arabidopsis plants | |

From the results reported in Table 1 and the Sequence Listing, it can be seen that the nucleotides/polypeptides of the inventions are useful, depending upon the respective individual sequence, to make plants with modified growth and phenotype characteristics, including:
a. modulated plant size, including increased and decreased height or length;
b. modulated vegetative growth (increased or decreased);
c. modulated organ number;
d. increased biomass;
e. sterility;
f. seedling lethality;
g. accelerated crop development or harvest;
h. accelerated flowering time;
i. delayed flowering time;
j. delayed senescence;
k. enhanced drought or stress tolerance;
l. increased chlorophyll and photosynthetic capacity;
m. increased anthocyanin content;
n. increased root growth, and increased nutrient uptake;
o. increased or decreased seed weight or size, increased seed carbon or nitrogen content;
p. modified, including increased, seed/fruit yield or modified fruit content;
q. enhanced foliage;
r. usefulness for making nutratceuticals/pharmaceuticals in plants;
s. plant lethality;
t. decrease seed fiber content to provide increased digestability;
u. modified ornamental appearance with modified leaves, flowers, color or foliage;
v. modified sterility in plants;
w. enhanced ability to grow in shade;
x. enhanced biotic stress tolerance;
y. increased tolerance to density and low fertilizer;
z. enhanced tolerance to high or low pH, to low or high nitrogen or phosphate;
aa. enhanced tolerance to oxidative stress;
bb. enhanced chemical composition;
cc. altered leaf shape;
dd. enhanced abiotic stress tolerance;
ee. increased tolerance to cold stress;
ff. increased starch content;
gg. reduced number or no seeds;
hh. enhanced plant strength;
ii. modified flower length;
jj. longer inflorescences;
kk. modified seed fiber content;
ll. modified fruit shape;
mm. modified fruit composition;
nn. modified seed yield;
oo. modified plant architecture, such as modified amount or angle of branching, modified leaf structure, or modified seed structure; and
pp. enhanced shade avoidance.

### Example 1

### Lead 80 (ME08386); Clone 733804 SEQ ID NO. 94

Lead 80 (SEQ ID NO. 94) encodes a 92 amino acid bHLH transcription factor from wheat. Plants transformed with this sequence were found to exhibit:

Enhanced growth, particularly on low-nitrate-containing media;

Enhanced photosynthesis on low-nitrate containing media;

Elongated hyocotyls, narrow leaves and often a flattened inflorescence.

Clone 733804 encodes a bHLH transcription factor that confers increased growth and improved photosynthetic efficiency on plants experiencing nitrogen deficiency stress. Transcription factors often control the expression of multiple genes in a pathway. As such, Clone 733804 may be involved in controlling the expression of several genes in a pathway, such as carbon flux through the TCA cycle (Yanagisawa et al., 2004). A related Arabidopsis bHLH transcription factor and potential ortholog (60% identity; clone 8607) is also able to confer a similar low nitrogen stress phenotype. Since the gain-of-function phenotype of clones 733804 and clone 8607 is conserved between wheat and Arabidopsis, these genes can have direct application for improving nitrogen stress tolerance increasing nitrogen use efficiency, and enhancing seedling vigor in a broad range of crops.

### Materials and Methods:

### Generation and Phenotypic Evaluation of T₁ Events.

Wild-type Arabidopsis Wassilewskija (Ws) plants were transformed with a Ti plasmid containing Clone 733804 in the sense orientation relative to the 35S promoter, as described above. The Ti plasmid vector used for this construct, CRS 338, contains the Ceres-constructed, plant selectable marker gene phosphinothricin acetyltransferase (PAT) which confers herbicide resistance to transformed plants. Ten independently transformed events were selected and evaluated for their qualitative phenotype in the T₁ generation.

The procedure for 1) identifying the candidate from a low nitrate tolerance superpool screen, 2) confirming the phenotype in the second and third generations and 3) determining the lack of significant negative phenotypes was as described below.

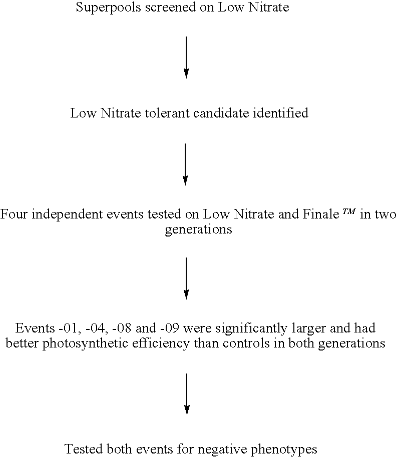

### Screening Superpools 62-71 for Tolerance to Low Nitrate Growth Conditions.

Two thousand seeds each from Superpools 62-71 were pooled together and plated on low nitrate media. DNA was isolated from each candidate plant and sequenced to determine which transgene was present.

### Growth Conditions and Planting Schema for Me08386 Under Low Nitrate Growth Conditions.

Evaluation of tolerance to low nitrate conditions (300 μM KNO₃ MS media) was done using five T₂ events (-01, -03, -04, -08 and -09). Subsequently, T₃ generation seeds for all five events were evaluated under low nitrate conditions.

### Results:

ME08386 was Identified from a Superpool Screen for Seedling Tolerance to Low Nitrate Conditions.

Superpools 62-71 were screened for seedlings that were larger or greener than controls on low nitrate growth media. Transgene sequence was obtained for 19 candidate seedlings. Two of the 19 candidate sequences BLASTed to ME08386.

### Four Events of ME08386 Show 3:1 Segregation for Finale™ Resistance.

Events -01, -04, -08 and -09 segregated 3:1 (R:S) for Finale™ resistance in the T₂ generation (data not shown).

### Four Events of ME08386 Showed Significantly Increased Growth Under Low Nitrate Growth Conditions in Both Generations.

Five events of ME08386 were sown as described in the Low Nitrate Assay in both the T₂ and the T₃ generations. In this study the seedling area at 14 days for transgenic plants within an event was compared to the seedling area for non-transgenic segregants pooled across the same plate. Four events, -01, -04, -08 and -09, were significant in both generations at p=0.05, using a one-tailed t-test assuming unequal variance (Table 1-1).

TABLE 1-1
T-test comparison of seedling area between transgenic seedlings and
pooled non-transgenic segregants after 14 days of growth on low nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME08386 | ME08386-01 | 0.073 | 29 | 0.056 | 17 | 0.00132 |
| ME08386 | ME08386- | 0.086 | 32 | 0.056 | 17 | 9.83E−07 |
| | 01-99 | | | | | |
| ME08386 | ME08386-04 | 0.067 | 32 | 0.056 | 18 | 9.22E−05 |
| ME08386 | ME08386- | 0.078 | 29 | 0.056 | 18 | 3.74E−06 |
| | 04-99 | | | | | |
| ME08386 | ME08386-08 | 0.064 | 27 | 0.053 | 20 | 0.0105 |
| ME08386 | ME08386- | 0.087 | 29 | 0.053 | 20 | 1.54E−09 |
| | 08-99 | | | | | |
| ME08386 | ME08386-09 | 0.065 | 30 | 0.057 | 19 | 0.00549 |
| ME08383 | ME08386- | 0.072 | 32 | 0.057 | 19 | 9.57E−07 |
| | 09-99 | | | | | |

### Four Events of ME08386 Showed Significantly Increased Photosynthetic Efficiency Under Low Nitrate Growth Conditions in Both Generations.

Five events of ME08386 were sown as described in the Low Nitrate Assay in both the T₂ and the T₃ generations. In this study, the seedling photosynthetic efficiency was measured as Fv/Fm comparing transgenic plants within an event to non-transgenic segregants pooled across the same plate. Four events, -01, -04, -08 and -09, were significant in both generations at p=0.05, using a one-tailed t-test assuming unequal variance (Table 1-2).

TABLE 1-2
T-test comparison of seedling photosynthetic efficiency between
transgenic seedlings and pooled non-transgenic segregants after
14 days of growth on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME08386 | ME08386-01 | 0.59 | 29 | 0.56 | 17 | 0.00982 |
| ME08386 | ME08386-01-99 | 0.64 | 32 | 0.56 | 17 | 8.76E−07 |
| ME08386 | ME08386-04 | 0.61 | 31 | 0.56 | 19 | 0.00338 |
| ME08386 | ME08386-04-99 | 0.61 | 28 | 0.56 | 19 | 0.00402 |
| ME08386 | ME08386-08 | 0.62 | 28 | 0.52 | 21 | 4.69E−05 |
| ME08386 | ME08386-08-99 | 0.63 | 26 | 0.52 | 21 | 1.59E−09 |
| ME08386 | ME08386-09 | 0.58 | 30 | 0.51 | 18 | 0.000819 |
| ME08383 | ME08386-09-99 | 0.58 | 32 | 0.51 | 18 | 0.000357 |

### Qualitative Analysis of the T₁ Plants:

The physical appearance of four of the ten T₁ plants was identical to the controls. Events -01, -03, -04, -08, -09 and -10 were noted as having flat inflorescences, but were still fully fertile.

### Qualitative and Quantitative Analysis of the T₂ Plants (Screening for Negative Phenotypes):

Events 01, -04, -08 and -09 of ME08386 exhibited no statistically relevant negative phenotypes. All four events showed the same flat inflorescence phenotype as noted in the T₁ generation, but this phenotype does not negatively affect yield. The plants also had slightly elongated hypocotyls and rosette leaves. The plants exhibited slightly elongated hypocotyls, elongated rosette leaves and flat bolts. But exhibited no observable or statistical differences between experimentals and controls with respect to germination rate, days to flowering, rosette after 7 days post-bolting, or fertility (silique number and seed fill).

### Example 2

### Lead 81 (ME03973) Clone 8607 SEQ ID NO. 96

Lead 81 (SEQ ID NO. **) encodes a 94 amino acid bHLH transcription factor from Arabidopsis. Plants transformed with this sequence were found to exhibit:

Enhanced growth, particularly on low-nitrate-containing media;

Enhanced photosynthesis on low-nitrate containing media;

Elongate hypocotyls, narrow leaves and often a flattened inflorescence.

Clone 8607 encodes an Arabidopsis basic-helix-loop-helix transcription factor. The clone was placed in the cDNA misexpression pipeline to test its utility in improving plant performance under various stress conditions. The gene is differentially expressed in heat, drought, and nitrogen-deficiency stress experiments and, therefore, can play a role in regulating genes important for stress tolerance or adaptation.

Clone 8607 encodes a bHLH transcription factor that confers increased growth and improved photosynthetic efficiency on plants experiencing nitrogen deficiency stress. Transcription factors often control the expression of multiple genes in a pathway. Clone 8607 may be involved in controlling the expression of several genes in a pathway, such as carbon flux through the TCA cycle (Yanagisawa et al., 2004). The function of clone 8607 is not known, but its regulation by nitrogen stress indicates it can function in plant responses to nitrogen deficiency. A related wheat bHLH transcription factor and potential ortholog (60% identity; clone 733804) is also able to confer a similar low nitrogen stress phenotype. Since the gain of function phenotype of clones 733804 and clone 8607 is conserved between wheat and Arabidopsis, these genes can have direct application for improving nitrogen stress tolerance and increasing nitrogen use efficiency and enhancing seedling vigor in a broad range of crops.

### Materials and Methods:

### Generation and Phenotypic Evaluation of T₁ Events.

Wild-type Arabidopsis Wassilewskija (Ws) plants were transformed with a Ti plasmid containing Clone 8607 in the sense orientation relative to the 35S promoter, as described above. The Ti plasmid vector used for this construct, CRS 338, contains the Ceres-constructed, plant selectable marker gene phosphinothricin acetyltransferase (PAT) which confers herbicide resistance to transformed plants. Five independently transformed events were selected and evaluated for their qualitative phenotype in the T₁ generation as per Ceres SOP 5-HTP T1 Plant Phenotyping.

The procedure for 1) identifying the candidate from a low nitrate tolerance superpool screen, 2) confirming the phenotype in the second and third generations and 3) determining the lack of significant negative phenotypes was as described below.

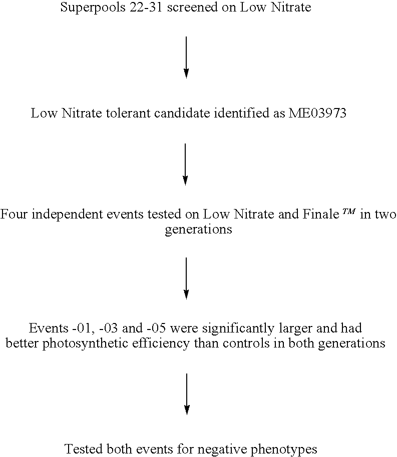

### Screening Superpools 22-31 for Tolerance to Low Nitrate Growth Conditions.

Two thousand seeds each from Superpools 22-31 were pooled together and plated on low nitrate media. DNA was isolated from each candidate plant and sequenced to determine which transgene was present.

### Growth Conditions and Planting Schema for ME03973 Under Low Nitrate Growth Conditions.

Evaluation of tolerance to low nitrate conditions (300 μM KNO₃ MS media) was done using four T₂ events (-01, -02, -03 and -05). Subsequently, T₃ generation seeds for all four events were evaluated under low nitrate conditions.

### Results:

ME03973 was Identified from a Superpool Screen for Seedling Tolerance to Low Nitrate Conditions.

Superpool 27 was screened for seedlings that were larger or greener than controls on low nitrate growth media. Line ME03973 was identified from among the candidates.

Superpools 22-31 were screened for seedlings that were larger or greener than controls on low nitrate growth media. Transgene sequence was obtained for 39 candidate seedlings. One of the 39 candidate sequences BLASTed to ME03973.

### Three Events of ME03973 Show 3:1 Segregation for Finale™ Resistance.

Events -01, -03 and -05 segregated 3:1 (R:S) for Finale™ resistance in the T₂ generatio.

### Three Events of ME03973 Showed Significantly Increased Growth Under Low Nitrate Growth Conditions in Both Generations.

Four events of ME03973 were tested on the Low Nitrate Assay in both the T₂ and the T₃ generations. In this study, the seedling area at 14 days for transgenic plants within an event was compared to the seedling area for non-transgenic segregants pooled across the same plate. Three events, -01, -03 and -05, were significant in both generations at p=0.05, using a one-tailed t-test assuming unequal variance (Table 2-1).

TABLE 2-1
T-test comparison of seedling area between transgenic seedlings and
pooled non-transgenic segregants after 14 days of growth on low nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME03973 | ME03973-01 | 0.077 | 27 | 0.055 | 17 | 2.35E−08 |
| ME03973 | ME03973- | 0.083 | 35 | 0.055 | 17 | 3.20E−10 |
| | 01-99 | | | | | |
| ME03973 | ME03973-03 | 0.073 | 33 | 0.056 | 11 | 0.000118 |
| ME03973 | ME03973- | 0.086 | 33 | 0.056 | 11 | 6.05E−06 |
| | 03-99 | | | | | |
| ME03973 | ME03973-05 | 0.069 | 29 | 0.061 | 20 | 0.0101 |
| ME03973 | ME03973- | 0.077 | 29 | 0.061 | 20 | 1.69E−05 |
| | 05-99 | | | | | |

### Three Events of ME03973 Showed Significantly Increased Photosynthetic Efficiency Under Low Nitrate Growth Conditions in Both Generations.

Four events of ME03973 were tested on the Low Nitrate Assay in both the T₂ and the T₃ generations. In this study, the seedling photosynthetic efficiency was measured as Fv/Fm comparing transgenic plants within an event to non-transgenic segregants pooled across the same plate. Three events, -01, -03 and -05, were significant in both generations at p=0.05, using a one-tailed t-test assuming unequal variance (Table 2-2).

TABLE 2-2
T-test comparison of seedling photosynthetic efficiency between
transgenic seedlings and pooled non-transgenic segregants after
14 days of growth on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME03973 | ME03973-01 | 0.61 | 27 | 0.56 | 17 | 0.000812 |
| ME03973 | ME03973-01-99 | 0.63 | 35 | 0.56 | 17 | 9.38E−06 |
| ME03973 | ME03973-03 | 0.61 | 33 | 0.54 | 12 | 0.00131 |
| ME03973 | ME03973-03-99 | 0.63 | 33 | 0.54 | 12 | 4.11E−05 |
| ME03973 | ME03973-05 | 0.61 | 29 | 0.54 | 20 | 0.000375 |
| ME03973 | ME03973-05-99 | 0.63 | 26 | 0.54 | 20 | 8.18E−06 |

### Qualitative Analysis of the T₁ Plants:

The noted physical appearance of the ten plants was identical to the controls. However, it is very likely that the elongated hypocotyls and rosette leaves, and flattened inflorescence was phenotype was present in the T₁ plants, but too subtle to be noted.

### Qualitative and Quantitative Analysis of the T₂ Plants (Screening for Negative Phenotypes):

Events -01, -03 and -05 of ME03973 exhibited no statistically relevant negative phenotypes. However, all events showed a flat inflorescence phenotype as noted in the T₁ generation, but this phenotype does not negatively affect yield. The plants also had slightly elongated hypocotyls and rosette leaves. The plants exhibited These events had slightly elongated hypocotyls, elongated rosette leaves and flat bolts, but exhibited no observable or statistical differences between experimentals and controls with respect to germination rate, days of the flowering, rosette area 7 days post-bolting, or fertility (silique number and seed fill).

### Example 3

### Lead 113 (ME08317); Clone 560948 SEQ ID NO. 90

| Construct | Event/Generation | Plant Stage | Assay | Result |
|---|---|---|---|---|
| 35S::560948 | -01/T₂ segregating | Seedling | Low | Significant |
| | plants | | Nitrate | at p ≦ .05 |
| | | | Tolerance | |
| 35S::560948 | -05/T₂ segregating | Seedling | Low | Significant |
| | plants | | Nitrate | at p ≦ .05 |
| | | | Tolerance | |
| 35S::560948 | -01/T₃ segregating | Seedling | Low | Significant |
| | plants | | Nitrate | at p ≦ .05 |
| | | | Tolerance | |
| 35S::560948 | -05/T₃ segregating | Seedling | Low | Significant |
| | plants | | Nitrate | at p ≦ .05 |
| | | | Tolerance | |

Ectopic expression of Clone 560948 under the control of the 35S promoter results in enhanced growth on low nitrate-containing media after 14 days compared to controls.

### ME08317 is Homologous to Leads 80 & 81.

ME08317 was identified from a reciprocal BLAST algorithm as having between 60-70% identity to Leads 80 & 81.

### One Event of ME08317 Segregates for a Single Insert, While the Other Event Segregates for 2 Inserts.

Event -01 segregated 3:1 (R:S) for Finale™ resistance in the T₂ generation. Event -05 segregated 15:1 (R:S) (data not shown).

### Two Events of ME08317 Showed Significantly Enhanced Growth Under Low Nitrate Growth Conditions in Both Generations.

Seeds representing two events of ME08317 from each of the T₂ and the T₃ generations were sown under conditions described in the Low Nitrate Assay. Both events, -01 and -05, showed a significant increase in growth in both generations at p=0.05 as measured using a one-tailed t-test and assuming unequal variance (Table 3-1).

TABLE 3-1
T-test comparison of seedling area between transgenic seedlings
and pooled non-transgenic segregants after 14 days of growth on
low nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME08317 | ME08317-01 | 0.076 | 41 | 0.058 | 32 | 6.8 × 10⁻⁷ |
| | (T₂) | | | | | |
| ME08317 | ME08317-01 | 0.074 | 27 | 0.058 | 32 | 1.7 × 10⁻⁸ |
| | (T₃) | | | | | |
| ME08317 | ME08317-05 | 0.081 | 47 | 0.055 | 18 | 5.5 × 10⁻⁸ |
| | (T₂) | | | | | |
| ME08317 | ME08317-05 | 0.078 | 35 | 0.055 | 18 | 3.6 × 10⁻⁸ |
| | (T₃) | | | | | |

### Qualitative Analysis of the T₁ Plants:

All events appeared wild-type. It is possible the T₂ morphological phenotype below was present in the T₁ generation, but too subtle to be noted.

### Qualitative and Quantitative Analysis of the T₂ Plants:

Events -01 and -05 of ME08317 had flat inflorescences and slightly elongated hypocotyls and rosette leaves.

### Example 4

### Lead 114 (ME10686): Clone 336524 (SEQ ID NO: 82)

| Construct | Event/Generation | Plant Stage | Assay | Result |
|---|---|---|---|---|
| 35S::336524 | -01/T₃ segregating plants | Seedling | Low Ammonium | Significant at |
| | | | Nitrate Tolerance | p ≦ .05 |
| 35S::336524 | -08/T₂ segregating plants | Seedling | Low Ammonium | Significant at |
| | | | Nitrate Tolerance | p ≦ .05 |
| 35S::336524 | -01/T₄ segregating plants | Seedling | Low Ammonium | Significant at |
| | | | Nitrate Tolerance | p ≦ .05 |
| 35S::336524 | -08/T₃ segregating plants | Seedling | Low Ammonium | Significant at |
| | | | Nitrate Tolerance | p ≦ .05 |

Ectopic expression of Clone 336524 under the control of the 35S promoter results in enhanced growth on low nitrate-containing media after 14 days compared to controls.

### ME10686 is Homologous to Leads 80 & 81.

ME10686 was identified from a reciprocal BLAST algorithm as having approximately 60% identity to Leads 80 & 81.

### Two Events of ME10686 Segregate for a Single Insert.

Events -01 and -08 segregated 3:1 (R:S) for Finale® resistance in the T₂ generation (data not shown).

### Two Events of ME10686 Showed Significantly Enhanced Growth Under Low Ammonium Nitrate Growth Conditions in Both Generations.

Seeds representing two events of ME10686 from each of the T₂ and the T₃ generations (or T₃ and T₄ generations, as is the case for Event -01) were sown under conditions described in the Low Ammonium Nitrate Assay. Both events, -01 and -08, showed a significant increase in growth in both generations at p=0.05 as measured using a one-tailed t-test and assuming unequal variance (Table 4-1).

TABLE 4-1
T-test comparison of seedling area between transgenic seedlings
and pooled non-transgenic segregants after 14 days of growth on
low ammonium nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME10686 | ME10686-01 | 0.081 | 44 | 0.063 | 6 | 1.36 × 10⁻⁵ |
| | (T₃) | | | | | |
| ME10686 | ME10686-01 | 0.078 | 50 | 0.063 | 6 | 9.06 × 10⁻⁵ |
| | (T₄) | | | | | |
| ME10686 | ME10686-08 | 0.111 | 31 | 0.084 | 20 | 5.73 × 10⁻⁴ |
| | (T₂) | | | | | |
| ME10686 | ME10686-08 | 0.103 | 41 | 0.084 | 20 | 1.15 × 10⁻³ |
| | (T₃) | | | | | |

### Qualitative Analysis of the T₁ Plants:

All events appeared wild-type. It is possible the T₂ morphological phenotype below was present in the T₁ generation, but too subtle to be noted.

### Qualitative and Quantitative Analysis of the T₂ Plants:

Events -01 and -08 of ME08317 flat inflorescences and slightly elongated hypocotyls and rosette leaves.

### Example 5

### Lead ME08328; Clone 560681 (SEQ ID NO:88)

| Construct | Event/Generation | Plant Stage | Assay | Result |
|---|---|---|---|---|
| 35S::560681 | -05/T₂ segregating plants | Seedling | Low Ammonium | Significant at |
| | | | Nitrate Tolerance | p ≦ .05 |
| 35S::560681 | -05/T₂ segregating plants | Seedling | Low Nitrate | Significant at |
| | | | Tolerance | p ≦ .05 |

### ME08328 is Homologous to Leads 80 & 81.

ME08328 was identified from a reciprocal BLAST algorithm as having approximately 70% identity to Leads 80 & 81.

### ME08328-05 Segregates for a Single Insert.

Event -05 segregated 3:1 (R:S) for Finale® resistance in the T₂ generation (data not shown).

### One Event of ME08328 Showed Significantly Enhanced Growth Under Both Low Ammonium Nitrate and Low Nitrate Growth Conditions.

Seeds representing one event of ME08328 were sown under conditions described in the Low Ammonium Nitrate and Low Nitrate Assays. Event -05 showed a significant increase in growth in both generations at p=0.05 as measured using a one-tailed t-test and assuming unequal variance (Tables 5-1 and 5-2).

TABLE 5-1
T-test comparison of seedling area between transgenic seedlings
and pooled non-transgenic segregants after 17 days of growth on
low ammonium nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME08328 | ME08328-05 | 0.107 | 16 | 0.081 | 25 | 5.11 × 10⁻⁵ |
| | (T₂) | | | | | |

TABLE 5-2
T-test comparison of seedling area between transgenic seedlings and
pooled non-transgenic segregants after 17 days of growth on low nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME08328 | ME08328-05(T₂) | 0.069 | 14 | 0.057 | 18 | 0.048 |

### Qualitative Analysis of the T₁ Plants:

All events appeared wild-type.

### Example 6

### Lead ME01905: Clone 4734 (SEQ ID NO: 84)

| Construct | Event/Generation | Plant Stage | Assay | Result |
|---|---|---|---|---|
| 35S::4734 | -03/T₂ segregating | Seedling | Low | Significant |
| | plants | | Ammonium | at p ≦ .05 |
| | | | Nitrate | |
| | | | Tolerance | |
| 35S::4734 | -05/T₂ segregating | Seedling | Low | Significant |
| | plants | | Ammonium | at p ≦ .05 |
| | | | Nitrate | |
| | | | Tolerance | |
| 35S::4734 | -03/T₂ segregating | Seedling | Low Nitrate | Significant |
| | plants | | Tolerance | at p ≦ .05 |
| 35S::4734 | -05/T₂ segregating | Seedling | Low Nitrate | Significant |
| | plants | | Tolerance | at p ≦ .05 |

ME01905 is homologous to Leads 80 & 81.

ME01905 was identified from a reciprocal BLAST algorithm as having approximately 60% identity to Leads 80 & 81.

### Two Events of ME01905 Show 3:1 Segregation for Finale™ Resistance.

Events -03 and -05 segregated 3:1 (R:S) for Finale™ resistance in the T₂ generation (data not shown).

### Two Events of ME01905 Showed Significantly Enhanced Growth Under Both Low Ammonium Nitrate and Low Nitrate Growth Conditions.

Seeds representing two events of ME01905 were sown under conditions described in the Low Ammonium Nitrate and Low Nitrate Assays. Events -03 and -05 showed a significant increase in growth in both generations at p=0.05 as measured using a one-tailed t-test and assuming unequal variance (Tables 6-1 and 6-2).

TABLE 6-1
T-test comparison of seedling area between transgenic seedlings and
pooled non-transgenic segregants after 17 days of growth on low
ammonium nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME01905 | ME08328-03(T₂) | 0.119 | 13 | 0.088 | 34 | 0.0012 |
| ME01905 | ME08328-05(T₂) | 0.107 | 17 | 0.096 | 32 | 0.0041 |

TABLE 6-2
T-test comparison of seedling area between transgenic seedlings and
pooled non-transgenic segregants after 17 days of growth on low nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME01905 | ME08328-03 | 0.086 | 12 | 0.058 | 31 | 1.58 × 10⁻⁵ |
| | (T₂) | | | | | |
| ME01905 | ME08328-05 | 0.075 | 17 | 0.06 | 33 | 8.91 × 10⁻⁵ |
| | (T₂) | | | | | |

### Qualitative Analysis of the T₁ Plants:

Events -01, -02, -03 and -05 had flat inflorescences, but were still fully fertile. Event -03 was also noted as having a glossy appearance.

### Qualitative and Quantitative Analysis of the T₂ Plants:

Events -01, -02, -03 and -05 of ME01905 had flat inflorescences and slightly elongated hypocotyls and rosette leaves. Events -01, -03 and -05 had a smaller rosette size and less seed yield compared to the control. Event -02 had a normal rosette size and seed yield.

### Example 7

### Lead ME01770; Clone 519 (SEQ ID NO: 86)

| Construct | Event/Generation | Plant Stage | Assay | Result |
|---|---|---|---|---|
| 35S::519 | -02/T₃ segregating | Seedling | Low | Significant |
| | plants | | Ammonium | at p ≦ .05 |
| | | | Nitrate | |
| | | | Tolerance | |
| 35S::4734 | -07/T₃ segregating | Seedling | Low | Significant |
| | plants | | Ammonium | at p ≦ .05 |
| | | | Nitrate | |
| | | | Tolerance | |
| 35S::519 | -02/T₃ segregating | Seedling | Low Nitrate | Significant |
| | plants | | Tolerance | at p ≦ .05 |
| 35S::4734 | -07/T₃ segregating | Seedling | Low Nitrate | Significant |
| | plants | | Tolerance | at p ≦ .05 |

### ME01770 is Homologous to Leads 80 & 81.

ME01770 was identified from a reciprocal BLAST algorithm as having approximately 70% identity to Leads 80 & 81.]

### Two Events of ME01770 Show 3:1 Segregation for Finale™ Resistance.

Events -02 and -07 segregated 3:1 (R:S) for Finale™ resistance in the T₂ generation (data not shown).

### Two Events of ME01770 Showed Significantly Enhanced Growth Under Both Low Ammonium Nitrate and Low Nitrate Growth Conditions.

Seeds representing two events of ME01770 were sown under conditions described in the Low Ammonium Nitrate and Low Nitrate Assays. Events -02 and -07 showed a significant increase in growth in both generations at p=0.05 as measured using a one-tailed t-test and assuming unequal variance (Tables 7-1 and 7-2).

TABLE 7-1
T-test comparison of seedling area between transgenic seedlings and
pooled non-transgenic segregants after 17 days of growth on low
ammonium nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME01770 | ME01770-02 | 0.125 | 12 | 0.106 | 17 | 0.0025 |
| | (T₃) | | | | | |
| ME01770 | ME01770-07 | 0.121 | 11 | 0.088 | 34 | 1.74 × 10⁻⁴ |
| | (T₃) | | | | | |

TABLE 7-2
T-test comparison of seedling area between transgenic seedlings and
pooled non-transgenic segregants after 17 days of growth on
low nitrate.

| | | | | Pooled Non- | | |
|---|---|---|---|---|---|---|
| | | Transgenic | | Transgenics | | t-test |
| Line | Events | Avg (cm²) | n | Avg (cm²) | n | p-value |
| ME01770 | ME01770-02 | 0.115 | 12 | 0.083 | 11 | 5.85 × 10⁻⁴ |
| | (T₃) | | | | | |
| ME01770 | ME01770-07 | 0.083 | 10 | 0.058 | 31 | 0.0013 |
| | (T₃) | | | | | |

### Qualitative Analysis of the T₁ Plants:

Event -01 was small with a long hypocotyl and died before flowering. Events -08 and -09 had long hypocotyls and died before flowering. Events -03 and -04 were small. Events -02 and -05 had long hypocotyls. Events -06 and -07 were small with long hypocotyls.

### Qualitative and Quantitative Analysis of the T₂ Plants:

Events -02, -04, -05, -06 and -07 of ME01770 had flat inflorescences and slightly elongated hypocotyls and rosette leaves. These events also had smaller rosettes and less seed yield compared to controls.

### Example 8

### Lead ME21445; Clone 653656 (SEQ ID NO: 92)

| Construct | Generation | Plant Stage | Assay | Result |
|---|---|---|---|---|
| 326::653656 | T₁ plants | Seedling | Morphological | Significant |
| | | | Phenotyping | |

### ME21445 is Homologous to Leads 80 & 81.

ME21445 was identified from a reciprocal BLAST algorithm as having approximately 80% identity to Leads 80 & 81.

Multiple Events of ME21445 Showed Significantly Enhanced Growth as T₁ Seedlings, with No Apparent Negative Phenotypes.

Transformed seeds containing the 326::653656 construct were sown under conditions described in the High Throughput Screening—T1 Generation protocol. Multiple seedlings/events appeared much larger than the control, but exhibited no apparent negative phenotypes, such as reduced rosette size or seed yield, as mature plants.

### Example 9

### Lead ME20023; Genomic Locus At1226945 (SEQ ID NO: 80)

| | | Plant | | |
|---|---|---|---|---|
| Construct | Generation | Stage | Assay | Result |
| 35S::At1g26945 | T₁ plants | Seedling | Morphological | Significant |
| | | | Phenotyping | |

### ME20023 is Homologous to Leads 80 & 81.

ME20023 was identified from a reciprocal BLAST algorithm as having approximately 80% identity to Leads 80 & 81.

Multiple Events of ME21445 Showed Significantly Enhanced Growth as T₁ Seedlings, with Elongated Hypocotyls, Flat Inflorescences, and Oblong Leaves.

Transformed seeds containing the 35S::At1g26945construct were sown under conditions described in the High Throughput Screening—T1 Generation protocol. Multiple seedlings/events appeared much larger and with elongated hypocotyls compared to the control. The plants exhibited flat inflorescences and oblong leaves at maturity.

### Example 10

### Determination of Functional Homolog Sequences

The “Lead” sequences described in above Examples are utilized to identify functional homologs of the lead sequences and, together with those sequences, are utilized to determine a consensus sequence for a given group of lead and functional homolog sequences.

A subject sequence is considered a functional homolog of a query sequence if the subject and query sequences encode proteins having a similar function and/or activity. A process known as Reciprocal BLAST (Rivera et al, Proc. Natl Acad. Sci. USA, 1998, 95:6239-6244) is used to identify potential functional homolog sequences from databases consisting of all available public and proprietary peptide sequences, including NR from NCBI and peptide translations from Ceres clones.

Before starting a Reciprocal BLAST process, a specific query polypeptide is searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides are designated as a cluster.

The main Reciprocal BLAST process consists of two rounds of BLAST searches; forward search and reverse search. In the forward search step, a query polypeptide sequence, “polypeptide A,” from source species S^{A} is BLASTed against all protein sequences from a species of interest. Top hits are determined using an E-value cutoff of 10⁻⁵ and an identity cutoff of 35%. Among the top hits, the sequence having the lowest E-value is designated as the best hit, and considered a potential functional homolog. Any other top hit that had a sequence identity of 80% or greater to the best hit or to the original query polypeptide is considered a potential functional homolog as well. This process is repeated for all species of interest.

In the reverse search round, the top hits identified in the forward search from all species are used to perform a BLAST search against all protein or polypeptide sequences from the source species S^{A}. A top hit from the forward search that returned a polypeptide from the aforementioned cluster as its best hit is also considered as a potential functional homolog.

Functional homologs are identified by manual inspection of potential functional homolog sequences. Representative functional homologs are shown in FIG. 1. The Figure represents a grouping of a lead/query sequence aligned with the corresponding identified functional homolog subject sequences. Lead sequences and their corresponding functional homolog sequences are aligned to identify conserved amino acids and to determine a consensus sequence that contains a frequently occurring amino acid residue at particular positions in the aligned sequences, as shown in FIG. 1.

Each consensus sequence then is comprised of the identified and numbered conserved regions or domains, with some of the conserved regions being separated by one or more amino acid residues, represented by a dash (-), between conserved regions.

Useful polypeptides of the inventions, therefore, include each of the lead and functional homolog sequences shown in FIG. 1, as well as the consensus sequences shown in the Figure. The invention also encompasses other useful polypeptides constructed based upon the consensus sequence and the identified conserved regions. Thus, useful polypeptides include those which comprise one or more of the numbered conserved regions in each alignment table in FIG. 1, wherein the conserved regions may be separated by dashes. Useful polypeptides also include those which comprise all of the numbered conserved regions in FIG. 1, alternatively comprising all of the numbered conserved regions in an individual alignment table and in the order as depicted in FIG. 1. Useful polypeptides also include those which comprise all of the numbered conserved regions in the alignment table and in the order as depicted in FIG. 1, wherein the conserved regions are separated by dashes, wherein each dash between two adjacent conserved regions is comprised of the amino acids depicted in the alignment table for lead and/or functional homolog sequences at the positions which define the particular dash. Such dashes in the consensus sequence can be of a length ranging from length of the smallest number of dashes in one of the aligned sequences up to the length of the highest number of dashes in one of the aligned sequences.

Such useful polypeptides can also have a length (a total number of amino acid residues) equal to the length identified for a consensus sequence or of a length ranging from the shortest to the longest sequence in any given family of lead and functional homolog sequences identified in FIG. 1.

The present invention further encompasses nucleotides that encode the above described polypeptides, as well as the complements thereof, and including alternatives thereof based upon the degeneracy of the genetic code.

The invention being thus described, it will be apparent to one of ordinary skill in the art that various modifications of the materials and methods for practicing the invention can be made. Such modifications are to be considered within the scope of the invention as defined by the following claims.

TABLE 2 summarizes the sequences found in FIG. 1.

Each of the references from the patent and periodical literature cited herein is hereby expressly incorporated in its entirety by such citation.

### REFERENCES

(1) Zhang et al. (2004) Plant Physiol. 135:615.
(2) Salomon et al. (1984) EMBO J. 3:141.
(3) Herrera-Estrella et al. (1983) EMBO J. 2:987.
(4) Escudero et al. (1996) Plant J. 10:355.
(5) Ishida et al. (1996) Nature Biotechnology 14:745.
(6) May et al. (1995) Bio/Technology 13:486)
(7) Armaleo et al. (1990) Current Genetics 17:97.
(8) Smith. T. F. and Waterman, M. S. (1981) Adv. App. Math. 2:482.
(9) Needleman and Wunsch (1970) J. Mol. Biol. 48:443.
(10) Pearson and Lipman (1988) Proc. Natl. Acad. Sci. (USA) 85: 2444.
(11) Yamauchi et al. (1996) Plant Mol. Biol. 30:321-9.
(12) Xu et al. (1995) Plant Mol. Biol. 27:237.
(13) Yamamoto et al. (1991) Plant Cell 3:371.
(14) P. Tijessen, “Hybridization with Nucleic Acid Probes” In Laboratory Techniques in Biochemistry and Molecular Biology, P.C. vand der Vliet, ed., c. 1993 by Elsevier, Amsterdam.
(15) Bonner et al., (1973) J. Mol. Biol. 81:123.
(16) Sambrook et al., Molecular Cloning: A Laboratory Manual, Second Edition, Cold Spring Harbor Laboratory Press, 1989, New York.
(17) Shizuya et al. (1992) Proc. Natl. Acad. Sci. USA, 89: 8794-8797.
(18) Hamilton et al. (1996) Proc. Natl. Acad. Sci. USA, 93: 9975-9979.
(19) Burke et al. (1987) Science, 236:806-812.
(20) Sternberg N. et al. (1990) Proc Natl Acad Sci USA., 87:103-7.
(21) Bradshaw et al. (1995) Nucl Acids Res, 23: 4850-4856.
(22) Frischauf et al. (1983) J. Mol. Biol, 170: 827-842.
(23) Huynh et al., Glover NM (ed) DNA Cloning: A practical Approach, Vol. 1 Oxford: IRL Press (1985).
(24) Walden et al. (1990) Mol Cell Biol 1: 175-194.
(25) Vissenberg et al. (2005) Plant Cell Physiol 46:192.
(26) Husebye et al. (2002) Plant Physiol 128:1180.
(27) Plesch et al. (2001) Plant J 28:455.
(28) Weising et al. (1988) Ann. Rev. Genet., 22:421.
(29) Christou (1995) Euphytica, v. 85, n.1-3:13-27.
(30) Newell (2000)
(31) Griesbach (1987) Plant Sci. 50:69-77.
(32) Fromm et al. (1985) Proc. Natl. Acad. Sci. USA 82:5824.
(33) Paszkowski et al. (1984) EMBO J. 3:2717.
(34) Klein et al. (1987) Nature 327:773.
(35) Willmitzer, L. (1993) Transgenic Plants. In: iotechnology, A Multi-Volume Comprehensive treatise (H. J. Rehm, G. Reed, A. Puler, P. Stadler, eds., Vol. 2, 627-659, VCH Weinheim-New York-Base1-Cambridge).
(36) Crit. Rev. Plant. Sci. 4:1-46.
(37) Fromm et al. (1990) Biotechnology 8:833-844.
(38) Cho et al. (2000) Planta 210:195-204.
(39) Brootghaerts et al. (2005) Nature 433:629-633.
(40) Lincoln et al. (1998) Plant Mol. Biol. Rep. 16:1-4.
(41) Lacomme et al. (2001), “Genetically Engineered Viruses” (C.J.A. Ring and E.D. Blair, Eds). Pp. 59-99, BIOS Scientific Publishers, Ltd. Oxford, UK.

What is claimed is:

1. An isolated nucleic acid molecule comprising:
(a) a nucleotide sequence encoding an amino acid sequence that is at least 85% identical to any one of Leads 80, 81, 113, 114, ME08328, ME01905, ME21445, and ME20023, SEQ ID NOS. 94, 96, 90, 82, 88, 84, 86, 92 and 80, respectively;
(b) a nucleotide sequence that is complementary to any one of the nucleotide sequences according to paragraph (a);
(c) a nucleotide sequence according to any one of SEQ ID NOS. 94, 96, 90, 82, 88, 84, 86, 92 and 80;
(d) a nucleotide sequence that is in reverse order of any one of the nucleotide sequences according to (c) when read in the 5′ to 3′ direction;
(e) a nucleotide sequence that is an interfering RNA to the nucleotide sequence according to paragraph (a);
(f) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(f) a nucleotide sequence encoding any one of the amino acid sequences identified as Leads 80, 81, 113, 114, ME08328, ME01905, ME21445, and ME20023, corresponding to SEQ ID NOS. 95, 97, 91, 83, 89, 85, 87, 93 and 81, respectively; or
(g) a nucleotide sequence encoding any one of the lead, functional homolog or consensus sequences in FIG. 1.

2. A vector, comprising:
a) a first nucleic acid having a regulatory region encoding a plant transcription and/or translation signal; and
a second nucleic acid having a nucleotide sequence according to any one the nucleotide sequences of claim 1, wherein said first and second nucleic acids are operably linked.

3. A method of modulating plant size, modulating vegetative growth, modulating plant architecture, seedling vigor and/or modulating the plant biomass, said method comprising introducing into a plant cell an isolated nucleic acid comprising a nucleotide sequence selected from the group consisting of:
(a) a nucleotide sequence encoding an amino acid sequence that is at least 85% identical to any one of Leads 80, 81, 113, 114, ME08328, ME01905, ME21445, and ME20023, SEQ ID NOS. 94, 96, 90, 82, 88, 84, 86, 92 and 80, respectively;
(b) a nucleotide sequence that is complementary to any one of the nucleotide sequences according to paragraph (a);
(c) a nucleotide sequence according to any one of SEQ ID NOS. 94, 96, 90, 82, 88, 84, 86, 92 and 80;
(d) a nucleotide sequence that is in reverse order of any one of the nucleotide sequences according to (c) when read in the 5′ to 3′ direction;
(e) a nucleotide sequence that is an interfering RNA to the nucleotide sequence according to paragraph (a);
(f) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(f) a nucleotide sequence encoding any one of the amino acid sequences identified as Leads 80, 81, 113, 114, ME08328, ME01905, ME21445, and ME20023, corresponding to SEQ ID NOS. 95, 97, 91, 83, 89, 85, 87, 93 and 81, respectively; or
(g) a nucleotide sequence encoding any one of the lead, functional homolog or consensus sequences in FIG. 1, wherein said plant produced from said plant cell has modulated plant size, modulated vegetative growth, modulated plant architecture, modulated seeding vigor and/or modulated biomass as compared to the corresponding level in tissue of a control plant that does not comprise said nucleic acid.

4. The method according to claim 3, wherein said consensus sequence comprises one or more of the conserved regions identified in any one of the alignment tables in FIG. 1.

5. The method according to claim 4, wherein said consensus sequence comprises all of the conserved regions identified in FIG. 1.

6. The method according to claim 5, wherein said consensus sequence comprises all of the conserved regions and in the order identified in FIG. 1.

7. The method according to claim 6, wherein said conserved regions are separated by one or more amino acid residues.

8. The method according to claim 7, wherein each of said of one or more amino acids consisting in number and kind of the amino acids depicted in the alignment table for the lead and/or functional homolog sequences at the corresponding positions that.

9. The method according to claim 8, wherein said consensus sequence has a length in terms of total number of amino acids that is equal to the length identified for a consensus sequence in FIG. 1, or equal to a length ranging from the shortest to the longest sequence in FIG. 1.

10. The method of claim 3, wherein said difference is an increase in the level of plant size, vegetative growth, organ number, seedling vigor and/or biomass.

11. The method of claim 3, wherein said isolated nucleic acid is operably linked to a regulatory region.

12. The method of claim 11, wherein said regulatory region is a promoter selected from the group consisting of YP0092 (SEQ ID NO: 38), PT0676 (SEQ ID NO: 12), PT0708 (SEQ ID NO: 17), PT0613 (SEQ ID NO: 5), PT0672 (SEQ ID NO: 11), PT0678 (SEQ ID NO: 13), PT0688 (SEQ ID NO: 15), PT0837 (SEQ ID NO: 24), the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, and the barley hordein gene promoter.

13. The method of claim 11, wherein said regulatory region is a promoter selected from the group consisting of p326 (SEQ ID NO: 76), YP0144 (SEQ ID NO: 55), YP0190 (SEQ ID NO: 59), p13879 (SEQ ID NO: 75), YP0050 (SEQ ID NO: 35), p32449 (SEQ ID NO: 77), 21876 (SEQ ID NO: 1), YP0158 (SEQ ID NO: 57), YP0214 (SEQ ID NO: 61), YP0380 (SEQ ID NO: 70), PT0848 (SEQ ID NO: 26), and PT0633 (SEQ ID NO:7), the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter.

14. The method of claim 11, wherein said regulatory region is a promoter selected from the group consisting of ribulose-1,5-bisphosphate carboxylase (RbcS) promoters such as the RbcS promoter from eastern larch (Larix laricina), the pine cab6 promoter, the Cab-1 gene promoter from wheat, the CAB-1 promoter from spinach, the cab1R promoter from rice, the pyruvate orthophosphate dikinase (PPDK) promoter from corn, the tobacco Lhcb1*2 promoter, the Arabidopsis thaliana SUC2 sucrose-H+ symporter promoter, and thylakoid membrane protein promoters from spinach (psaD, psaF, psaE, PC, FNR, atpC, atpD, cab, rbcS, PT0535 (SEQ ID NO: 3), PT0668 (SEQ ID NO: 2), PT0886 (SEQ ID NO: 29), PRO924 (SEQ ID NO: 78), YP0144 (SEQ ID NO: 55), YP0380 (SEQ ID NO: 70) and PT0585 (SEQ ID NO: 4).

15. A plant cell comprising an isolated nucleic acid comprising a nucleotide sequence selected from the group consisting of:
(a) a nucleotide sequence encoding an amino acid sequence that is at least 85% identical to any one of Leads 80, 81, 113, 114, ME08328, ME01905, ME21445, and ME20023, corresponding to SEQ ID NOS. 94, 96, 90, 82, 88, 84, 86, 92 and 80, respectively;
(b) a nucleotide sequence that is complementary to any one of the nucleotide sequences according to paragraph (a);
(c) a nucleotide sequence according to any one of SEQ ID NOS. 94, 96, 90, 82, 88, 84, 86, 92 and 80;
(d) a nucleotide sequence that is in reverse order of any one of the nucleotide sequences according to (c) when read in the 5′ to 3′ direction;
(e) a nucleotide sequence that is an interfering RNA to the nucleotide sequence according to paragraph (a);
(f) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(f) a nucleotide sequence encoding any one of the amino acid sequences identified as Leads 80, 81, 113, 114, ME08328, ME01905, ME21445, and ME20023 corresponding to SEQ ID NOS. 95, 97, 91, 83, 89, 85, 87, 93 and 81, respectively; or
(g) a nucleotide sequence encoding any one of the lead, functional homolog or consensus sequences in FIG. 1 .

16. A transgenic plant comprising the plant cell of claim 15.

17. Progeny of the plant of claim 16, wherein said progeny has modulated plant size, modulated vegetative growth, modulated plant architecture, modulated seedling vigor and/or modulated biomass as compared to the corresponding level in tissue of a control plant that does not comprise said nucleic acid.

18. Seed from a transgenic plant according to claim 16.

19. Vegetative tissue from a transgenic plant according to claim 16.

20. A food product comprising vegetative tissue from a transgenic plant according to claim 16.

21. A feed product comprising vegetative tissue from a transgenic plant according to claim 16.

22. A product comprising vegetative tissue from a transgenic plant according to claim 16 used for the conversion into fuel or chemical feedstocks.

23. A method for detecting a nucleic acid in a sample, comprising:
providing an isolated nucleic acid according to claim 1;
contacting said isolated nucleic acid with a sample under conditions that permit a comparison of the nucleotide sequence of the isolated nucleic acid with a nucleotide sequence of nucleic acid in the sample; and
analyzing the comparison.

24. A method for promoting increased biomass in a plant, comprising:
(a) transforming a plant with a nucleic acid molecule comprising a nucleotide sequence encoding any one of the lead, functional homolog or consensus sequences in FIG. 1; and
(b) expressing said nucleotide sequence in said transformed plant, whereby said transformed plant has an increased biomass or enhance seedling vigor as compared to a plant that has not been transformed with said nucleotide sequence.

25. A method for modulating the biomass of a plant, said method comprising altering the level of expression in said plant of a nucleic acid molecule according to claim 1.
